(19) United States
(12) Patent Application Publication
(10) Pub. No.: US 2004/0171139 A1
(43) Pub. Date: Sep. 2, 2004

(54) Fabricated biofilm storage device

(76) Inventors: Angela M. Belcher, Lexington, MA (US); Seung-Wuk Lee, Albany, CA (US); Brent L. Iverson, Austin, TX (US); Soo-Kwan Lee, Boston, MA (US)

Correspondence Address: STEPHEN B MAEBIUS, FOLEY AND LARDNER, 3000 K STREET N W SUITE 500, WASHINGTON, DC, 20007-5109, US

(21) Appl. No.: 10/668,600

(22) Filed: Sep. 24, 2003

Related U.S. Application Data
(60) Provisional application No. 60/413,081, filed on Sep. 24, 2002.

(51) Int. Cl. A01N 1/02; C12M 1/34

(52) U.S. Cl. 435/287.1; 435/2

(57) ABSTRACT

The present invention includes a method and composition of storing and preserving biofilms for input and output of high-density information. One form of the present invention is a fabricated biofilm storage device with a biologic material applied to a substrate to form, e.g., a dry thin film stable at room temperature for extended periods of time. Another form of the present invention is a method of fabricating a biofilm storage device in which a biologic material is applied to a substrate under conditions that promote alignment of the biologic material on the substrate. The composition, method, and kit of the present invention have universal application in biologics, magnetics, optics and microelectronics.

### RELATED APPLICATIONS

This application claims priority to provisional application serial No. 60/413,081 to Lee et al. which is incorporated by reference herein in its entirety.

### STATEMENT OF FEDERAL GOVERNMENT RESEARCH SUPPORT

The U.S. Government may own certain rights in this invention, pursuant to the terms of the National Science Foundation and the Army Research Office, grant number DA 10-01-0456.

### FIELD OF THE INVENTION

The present invention is directed to the field of molecular storage devices in general, and specifically, toward the storage and preservation of fabricated biofilms for input and output of high-density information.

A nucleotide and/or amino acid sequence listing is incorporated by reference of the material on computer readable form.

### BACKGROUND OF THE INVENTION

The use of “biologic” materials to process the next generation of microelectronic devices provides a possible solution to resolving the limitations of traditional processing and memory methods. The critical factors in this approach towards the successful development of so-called organic-inorganic hybrid materials are identifying the appropriate compatibilities and combinations of biologic and inorganic materials, the synthesis and application of the appropriate materials, and the long-term storage of these biologic storage devices. The appropriate long-term storage of biologic materials is of enormous economic benefit, especially when it reduces weight and storage space and increases or preserves material stability.

Current technologies used to store biologic materials such as viruses and their products (e.g., DNA and proteins), or other biologic materials, are expensive and/or require extensive and cumbersome chemical modification techniques. Biologic materials, in general, are highly sensitive to their environment and require highly specific and often costly materials to ensure their stability, activity, and longevity. Few biologic materials are stable at room temperature for extensive periods of time. In fact, biologic materials are often considered unstable at room temperature. Viruses and bacteria, for example, are temperature and metabolite sensitive, require continuous feedings and appropriate air (gas) conditions to maintain activity, and must be frequently monitored for changes in growth and density.

For storage and preservation of biologic materials, several methods exist. Low temperature storage methods or freeze drying (e.g., suspending the materials in 10% glycerol at temperatures as low as −20 to −80 degrees Centigrade) or a poly (ethylene) glycol-modification technique are generally used. Dessication is another options that offers both advantages and disadvantages. While dessication is not as costly, it does not allow for large-scale preparations (i.e., industrial quantities). Freeze drying, on the other hand, may be used for large-scale production; however, the process is extremely damaging to sensitive biologic materials. Freeze drying is also very inconvenient, cannot ensure sterility and is very cost ineffective, as it requires that expensive agents (e.g., dry ice or other cooling agents) be used even when transferring materials from one facility to another.

There are several limitation to current method used for the preservation and storage of biologic materials. Present methods are not durable for prolonged periods, the recovery yields of the biologic materials after storage are often extremely low, and the quality and activity of the recovered biologic material is generally reduced. Therefore, there remains a need to provide long-term and cost-effective methods to store and preserve biologic materials while retaining material stability and or activity, and without losing large amounts of the material or its activity. Proper long-term storage is essential, especially where biologic materials are used as replacements for semiconductors, optical storage devices, and other microelectronic devices.

### SUMMARY OF THE INVENTION

The subject matter of the present invention includes the storage of variable density organic and inorganic information as a fabricated film that may be specifically engineered and custom designed. As used herein, biologic material film fabrication, also referred to as biofilms, may be used to store both organic and inorganic information from one or more biologic materials, wherein one or more biologic materials may be further bound to other organic or inorganic molecules. Applications of the present invention extend to medicine, engineering, computer technology and optics. Moreover the stored information may be biologic, electrical, magnetic, optical, microelectronic, mechanical and combinations thereof.

In one form, the present invention is a fabricated biofilm storage device comprising a substrate coated with a biologic material applied to a contacting surface to form a stable film.

Another form of the present invention is a method of fabricating a biofilm storage device that includes the steps of applying a biologic material to a substrate with a contacting surface that promotes uniform alignment of the biologic material on the contacting surface and allows the formation of a stable film.

In yet another form, the present invention is a kit for fabrication a biofilm storage device comprising a substrate with a surface and a biologic material capable of binding specifically to the surface to form a dry thin film.

Still another form of the present invention is a hybrid fabricated film storage device comprising a substrate comprising an inorganic material with a surface and a biologic material applied to the surface to form a stable and thin film, wherein the film may be biologically active or interact with biologic components.

### BRIEF DESCRIPTION OF THE DRAWINGS

The above and further advantages of the invention may be better understood by referring to the following description in conjunction with the accompanying drawings in which corresponding numerals in the different FIGURES refer to the corresponding parts in which:

FIG. 1 depicts (A) photograph of the biofilm, (B) polarized optical micrograph (POM) image of the biofilm, (C) atomic force microscopy (AFM) image of the individual M13 bacteriophage on the mica surface (contacting surface), and (D) surface morphology of the biofilm contacting surface in accordance with the present invention;

FIG. 2 depicts the relationship between the titer number and days showing the log plot of titer number and days since fabrication of the biofilm in accordance with the present invention;

FIG. 3 depicts selected random amino acid sequences in accordance with the present invention;

FIGS. 4A-C depict XPS spectra of structures in accordance with the present invention;

FIGS. 5A-E depicts phage recognition of heterostructures in accordance with the present invention;

FIGS. 6-10 depict specific amino acid sequences in accordance with the present invention;

FIGS. 11(A) and (B) depict schematic diagrams of the smectic alignment of M13 phages in accordance with the present invention;

FIGS. 12A-F depict the A7-ZnS suspensions: (A) and (B) POM images, (C) AFM image, (D) SEM image, (E) TEM image and (F) TEM image (with electron diffraction insert);

FIGS. 13A-F depict images of the M13 bacteriophage nanoparticle biofilm, including (A) photograph of the film, (B) schematic diagram of the film structure, (C) AFM image, (D) SEM image, (E) and (F) TEM images along the x-z and z-y planes;

FIG. 14 depicts the effect of glucose/sucrose and phage on β-galactosidase activity during storage at room temperature after (A) drying in desiccator, and (B) freeze-drying, where (-dark square) is β-galactosidase dried with sugar plus phage, (-dark triangle) is β-galactosidase dried with sugar, (-dark circle) is β-galactosidase dried with phage, and (-dark inverted triangle) is β-galactosidase without any additives and day 0 represents the recovered activity after freeze-drying or drying in desiccator; and

FIG. 15 illustrates confocal microscopy images of fluorescent GFPuv viral film one day after fabrication with GFPuv and phage, wherein variations in glucose:sucrose are (A) 5 mg/mL:50 mg/mL, (B) 2.5 mg/mL:25 mg/mL, and (C) no glucose or sucrose.

FIGS. 16A-C. (A) Photograph of M13 virus film. (B) Schematic diagram of the M13 virus film structure in the bulk which has a chiral smectic C ordering structure (z: director (molecular long axis); n: layer normal; θ: tilted angle; Φ: azimuthal rotation angle). (C) a schematic diagram of the surface morphology of the M13 virus film of which helical ordering structure is unwound and formed a zig-zag pattern due to surface effects. Dotted lines represent disclination lines and the spacing between two neighboring disclination lines correspond to half pitch (½P) of the chiral smectic C helical patterns.

FIGS. 17A-E. Chiral smectic C structure of the viral film from sample 1 (9.93 mg/ml). (A) POM image showing the dark and bright stripe patterns (36.8 pm) (scale bar: 100 μm; cross represents the direction of analyzer (A) and polarizer (P)), (B) SEM image of viral film showing zig-zag pattern dechiralization defects on the surface (scale bar: 50 μm). (C) AFM image of the viral film surface that shows the smectic C alignment. (scale bar: 1 μm), (D) TEM image of M13 virus (scale bar: 100 nm), and (E) a laser light diffraction pattern from the viral film.

FIGS. 18A-D. POM and AFM images showing distortion of the smectic structures and phase transitions from sample 1. (A) POM image showing the distorted dark and bright stripe patterns (scale bar: 100 μm), (B) POM image showing the phase transition (C), (D)AFM images corresponded to POM image (A) and (B) respectively.

FIGS. 19A-E. POM images of sample 7 that showed texture changes from a vertical stripe (A) pattern to horizontal stripe patterns (B). Smectic A morphologies of sample 10. (C) POM image showing the vertical stripe patterns (62.4 μm) (10× scale bar: 100 μm), (D) AFM image of the viral film surface showing the smectic A alignment. (scale bars: 1 μm), (E) SEM images of viral film surface showing the chevron-like cracked patterns and a high-resolution SEM image in the inset.

FIGS. 20A-B. Nematic morphologies of the viral film (sample 11). (A) POM image showing the crooked schlieren dark brush patterns (scale bar: 100 μm), (B) AFM images of viral film surface showing the nematic ordering of the smectic domains.

FIG. 21. A schematic diagram illustrating alignment of nanomaterials using an anti-streptavidin M13 virus and a streptavidin linker.

FIGS. 22A-D. (A) Photograph of virus pellet (i), streptavidin conjugated gold nanoparticles suspension (ii), and gold nanoparticle conjugated with virus (Au-virus) suspension (iii). (B) POM image of Au-virus suspension. (C) TEM image of a virus that bound to a 10 nm gold nanoparticle (scale bar: 100 nm) and a lattice fringe image and a fast Fourier transformation image of gold nanoparticle from the same TEM grids (insets, scale bar: 5 nm). (D) TEM image of Au-virus aggregations (scale bar: 500 nm).

FIGS. 23A-G. (A) Photograph of Au-virus film. (B) POM image of Au-virus film (scale bar: 20 μm) (C) SEM image of the Au-virus film surface morphology that shows the long range zig-zag patterns (scale bar: 5 μm). (D) AFM image of the Au-virus film (scale bar: 1 μm). (E) DIC image of Fluorescein-virus (F-virus) cast film (scale bar: 10 μm) (F) Fluorescence images of virus conjugated with fluorescein (F-virus) and (G) phycoerythrin (β-virus) cast films that show one micrometer fluorescent striped patterns (scale bars: 10 μm).

### DETAILED DESCRIPTION OF THE INVENTION

This application claims priority to provisional application serial No. 60/413,081 to Lee et al. which is incorporated by reference herein in its entirety, including the detailed description, the figures, the working examples, and the claims.

Also, U.S. Patent application Ser. No. 10/157,775 filed May 29, 2002 to Belcher et al. is hereby incorporated by reference in its entirety, as well as the provisional priority patent application 60/326,583 filed Oct. 2, 2001. In particular, working example II on biofilm preparation and characterization is incorporated by reference.

While the making and using of various embodiments of the present invention are discussed, it should be appreciated that the present invention provides many inventive concepts that may be embodied in a wide variety of specific contexts. The specific embodiments discussed herein are merely illustrative of ways to make and use the invention are not meant to limit the scope of the present invention in any way.

Terms used herein have meanings as commonly understood by a person of ordinary skill in the areas relevant to the present invention; Terms such as “a,” “an,” and “the” are not intended to refer to only a singular entity, but include the general class of which a specific example may be used for illustration. The terminology herein is used to describe specific embodiments of the invention, but their usage does not limit the invention, except as outlined in the claims. As used throughout the present specification, the terms “film” and “biofilm” are used interchangeably.

As used herein, the term “biologic material” refers to a virus, bacteriophage, bacteria, peptide, protein, amino acid, steroid, drug, chromophore, antibody, enzyme, single-stranded or double-stranded nucleic acid, vaccine, and any chemical modifications thereof. The biologic material may self-assemble to form a dry thin film on the contacting surface of a substrate. Dry thin films can be either substantially free of solvent so they are completely dry within conventional detection limits for dryness, or can be retaining residual solvent from the drying process so that the film is solid-like and self-supporting but still has residual wetness from solvent. In many cases, films can be left in a partially hydrated state, and the state of hydration can be optimized for a given application. Self-assembly may permit and random or uniform alignment of the biologic material on the surface. In addition, the biologic material may form a dry thin film that is externally controlled by solvent concentration, application of an electric and or magnetic field, optics, or other chemical or field interactions.

The term “inorganic molecule” or “inorganic compound” refers to compounds such as, e.g., indium tin oxide, doping agents, metals, minerals, radioisotope, salt, and combinations, thereof. Metals may include Ba, Sr, Ti, Bi, Ta, Zr, Fe, Ni, Mn, Pb, La, Li, Na, K, Rb, Cs, Fr, Be, Mg, Ca, Nb, Tl, Hg, Cu, Co, Rh, Sc, or Y. Inorganic compounds may include, e.g., high dielectric constant materials (insulators) such as barium strontium titanate, barium zirconate titanate, lead zirconate titanate, lead lanthanum titanate, strontium titanate, barium titanate, barium magnesium fluoride, bismuth titanate, strontium bismuth tantalite, and strontium bismuth tantalite niobate, or variations, thereof, known to those of ordinary skill in the art.

The term “organic molecule” or “organic compound” refers to compounds containing carbon alone or in combination, such as nucleotides, polynucleotides, nucleosides, steroids, DNA, RNA, peptides, protein, antibodies, enzymes, carbohydrate, lipids, conducting polymers, drugs, and combinations, thereof. A drug may include an antibiotic, antimicrobial, anti-inflammatory, analgesic, antihistamine, and any agent used therapeutically or prophylactically against mammalian pathologic (or potentially pathologic) conditions.

As used herein, a “substrate” may be a microfabricated solid surface to which molecules attach through either covalent or non-covalent bonds and includes, e.g., silicon, Langmuir-Bodgett films, functionalized glass, germanium, ceramic, a semiconductor material, PTFE, carbon, polycarbonate, mica, mylar, plastic, quartz, polystyrene, gallium arsenide, gold, silver, metal, metal alloy, fabric, and combinations thereof capable of having functional groups such as amino, carboxyl, thiol or hydroxyl incorporated on its surface. Similarly, the substrate may be an organic material such as a protein, mammalian cell, organ, or tissue with a surface to which a biologic material may attach. The surface may be large or small and not necessarily uniform but should act as a contacting surface (not necessarily in monolayer). The substrate may be porous, planar or nonplanar. The substrate includes a contacting surface that may be the substrate itself or a second layer (e.g., substrate or biologic material with a contacting surface) made of organic or inorganic molecules and to which organic or inorganic molecules may contact. The substrate can be cylindrical or non-flat. Substrates can be supported to improve their mechanical strength or surface to volume ratio. Arrays can be made. Macroporous beads can be used including glass and polystyrene beads. Dense packed pins can be used. Substrate surfaces can be grooved, micromachined, or otherwise made non-flat.

In general, the biofilm is created by applying a biologic material to the contacting surface of a substrate. The contact may be through a self-assembly of the biologic material or may be controlled by the surface itself or by external conditions such as solvent concentration, magnetic field, electric field, optics, and combinations thereof. In some cases, the substrate itself may serve as the contacting surface and may also control the nature and amount of biologic material contact. In other embodiments, the contacting surface may be a second substrate that may include one or more organic and or inorganic molecules applied to the contacting surface and to which the biologic material will be in contact.

The term “solvent” as used herein includes solutions of appropriate ionic strength to encourage high-density arrays or arrangements of the biologic material. The arrays may be ordered or random. When ordered, the solvent (with or without external control) concentration may be such to promote liquid crystal formation of the biologic material. The biologic material may be preincubated with the contacting surface and or with one or more organic or inorganic molecules. The preincubation may promote formation of particles in the nanometer scale. This preincubation may be further controlled by external conditions such as those described above.

All technical and scientific terms used herein have the same meaning as commonly understood by one of ordinary skill in the art to which this invention belongs, unless defined otherwise. Methods and materials similar or equivalent to those described herein may be used in the practice or testing of the present invention, the generally used methods and materials are now described.

Building and preserving well-ordered and well-controlled two- and three-dimensional structures at the nanolength scale is the major goal of building next generation optical, electronic and magnetic materials and devices. Many researchers and companies have focused on building such structures using only traditional materials (e.g. inorganic compounds). As disclosed herein, the present inventors have demonstrated that soft materials (e.g., organic and biologic materials) can act as self-organizers that assemble both organic and inorganic materials at the nanoscale level. Storage of these soft mixed materials, (organic and inorganic) however, has proven challenging.

The present invention provides cost-effective, long-term storage devices composed of soft mixed materials. There are several advantages to using the present invention in medical, engineering, material sciences and optical applications. The present invention includes several effects not readily resolved in earlier work. First, the dry thin film fabrication method requires few resources that are of minimal expense. In addition, the films are easy to store at they require little space and are amenable to room temperature conditions, and therefore is especially cost-effective. Moreover, the films require little effort to manufacture in large scale with little loss over time of activity, structure or other important properties. Finally, thin film fabrication of the present invention is a high-capacity storage device. For example, the biofilm fabricated with bacteriophage can store over 4×10¹³ viruses in a square centimeter of film.

The thickness of the thin film is not particularly limited but can be, for example, about 100 nm to about 100 microns, and more particularly about 500 nm to about 50 microns, and more particularly, about one micron to about 25 microns.

The inventors have previously shown that biologic materials such as peptides and bacteriophage can bind to semiconductor materials. These biologic materials were developed into nucleating nanoparticles that may direct their self-assembly with an ability to recognize and bind other organic and inorganic materials with face specificity, to nucleate size-constrained crystalline semiconductor materials, and to control the crystallographic phase of nucleated nanoparticles (Lee S-W, Mao C, Flynn CE, Belcher AM. Ordering of Quantum Dots Using Genetically Engineered Viruses. 2002 Science 296:892-895, relevant portions incorporated herein by reference in their entirety including description of self-supporting polymer films, and storage of viral films at room temperature for at least 7 months without loss of ability to infect bacterial host and with little loss of titer). Moreover, the aspect ratio of the nanoparticles can be controlled and, therefore, so can the electrical, magnetic, and optical properties. This binding of a biologic material to a surface or thin substrate (e.g., semiconductor material) forming an equally thin layer of the biologic material is referred to as a biofilm.

In general, a biofilm of the present invention may contain both organic and/or inorganic materials (or molecules). It may comprise a substrate, an organic layer, a second organic layer, and an inorganic layer or various combinations thereof. Each organic layer may comprise one or more different types of biologic and/or organic materials; similarly, each inorganic layer may comprise one or more different type of inorganic materials. Generally, the biofilm surface is well-ordered and may offer biologic, electrical, magnetic, and/or optical properties to the film enabling it to hold and store biologic, electrical, magnetic, and/or optical information.

In practice, biofilms have been defined as communities of biologic materials or microorganisms attached to a surface. Biofilm growth depends on the age of the biologic material or microorganism (e.g., culture), the build-up of potentially harmful (toxic) by-products or metabolites, and the consumption or use of other materials or nutrients for growth, stability or maintenance. Biofilms may be composed of natural or genetically engineered biologic materials. Of special interest is the use of biologic materials that self assemble. For example, bacteriophage that are genetically engineered to bind to other materials (e.g., semiconductor materials) also organize into well-ordered structures.

Thus, the self-assembling biological materials (e.g., bacteriophage) may be selected based on specific binding properties to particular surfaces and used to create well-ordered structures of the materials selected. These well-ordered structures may be further used to form layers and/or to support biologic, magnetic, optical, or electrical properties to the film. Thus, the biofilm may serve as an information storage device or optical storage media for memory, either of which may be used to store and read bits of data-data that is biologic, magnetic, optical, electrical and combinations thereof.

In supporting magnetic, optical, or electrical conditions, the present invention becomes a biologic material storage device with specific alignment properties. For example, an M13 bacteriophage that has specific binding properties is used to create a biofilm storage device in one of three liquid crystalline phases, a directional order in the nemetic phase, a twisted nemetic structure in the cholesteric phase, and both directional and positional order in smectic phase. The well-ordered biofilm storage device is, thus, created with biologic material alone or in combination with other organic or inorganic molecules (materials) to create, e.g., a type of thin film transistor.

In terms of chemical composition, a bacteriophage (or any virus or other biologic material of interest) is one type of natural “biopolymer” that can stick cohesively to itself and form a type of thin film surface. In general, the best biopolymers are those for which size and chemical composition may be controlled exactly, where one method of control is by genetic engineering. Controlled biopolymers offer precise known structure and composition. As a result, fabrication of the film using the controlled biopolymer may be specifically designed as needed. Bacteriophage, for example, are filamentous in shape (880 nm in length and 6.6 nm in width) with a surface covered by 2,700 copies of major protein units (known as pVIII). The following example describes the biofilm fabrication method, device and kit of the present invention.

Example of Biofilm Fabrication Storage Device

Viral films using the Ph. D. 12mer system obtained from New England Biolab that contained 10⁹ population of phage were amplified in large volume to get the highly concentrated viral suspension using previously described methods (J. Sambrook, E. F. Fritsch, T. Maniatis, Molecular Cloning A Laboratory Manual, Cold Spring Harbor Laboratory Press: New York, ed. 2, 1989). A 3.2 mL phage library suspension (concentration: at least 10⁹ phages/μL) and 4 mL of overnight culture were added to 400 mL LB medium and incubated for four and half hours in 37 degrees Centigrade. After purification of the phage, an approximately 30 mg pellets was obtained. The pellet was resuspended to 1 mL of Tris-buffered saline (TBS) at pH 7.5. This highly concentrated suspension (approximately 5 mg/mL) was used to fabricate the viral film.

The viral film was fabricated on the liquid/solid interfaces with gradient decrease of the liquid phase by evaporation of the solvent in a dessicator. As the solvent is gradually removed, the phage particles formed epitaxial layer domains on the surface of a solid substrate.

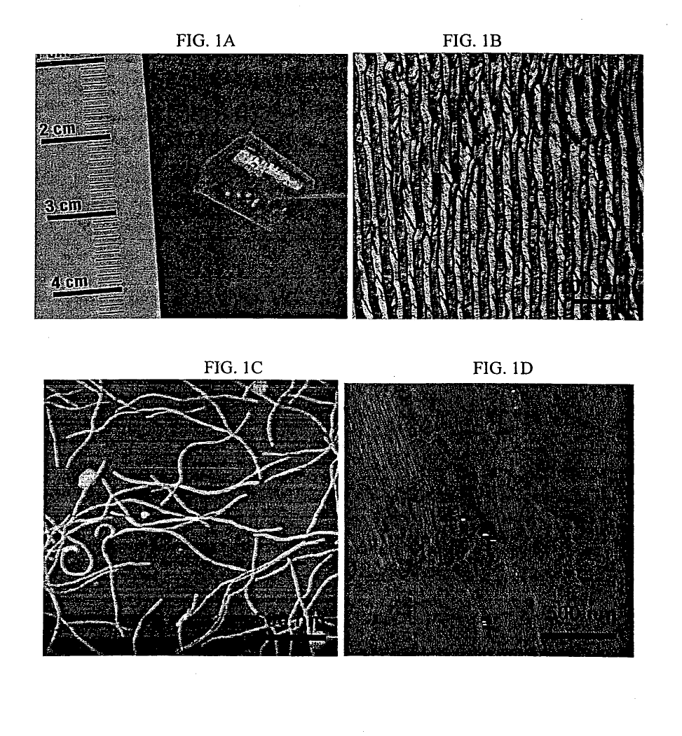

Poparized optical micrograph (POM) data of the phage layer that was formed showed in approximately 34 μm repeating patterns that continued to the centimeter scale. FIGS. 1B and C show the POM image of the viral film and AFM image of the individual phage particles, respectively. FIG. 1D shows an AFM image of the structure of the ordered viruses when assembled into a film.

It is clear that phage particles form an approximately 500 nm domaim. In addition, the phage particles are laterally stacked on each other. These lateral stacks form micro-domains that are packed to form a lamellar-like layer in the bulk film (see FIG. 1D). Sequences obtained from these particles are shown in TABLE 1.

TABLE 1

Sequence results from suspension before
screening.

| | | |
|---|---|---|
| Sample Number | Sequence | SEQ ID NO |

| | | |
|---|---|---|
| 2 | WQSELXXASNLP | SEQ ID NO:96 |
| 2 | AEATEARPYLRA | SEQ ID NO:97 |
| 3 | AYHNSGKTKTET | SEQ ID NO:98 |
| 4 | SPITPPLPPLPE | SEQ ID NO:99 |
| 5 | ETNLGPQPYPVR | SEQ ID NO:100 |
| 6 | SQLYNTPPQTAV | SEQ ID NO:101 |

Of importance is that the viral film preserves the original phage library in its entirety without losing its ability to infect. This is illustrated by resuspending the viral film and using it to biologically pan (biopan) for the streptaven target-a target known to have specific binding motifs, such as His-Pro-Gln. After the second round of sequencing the results show that the His-Pro-Gln sequence appears at the end of the pIII units. After the fourth round screening, all peptide sequences are found to exhibit the consensus sequence, His-Pro-Gln.

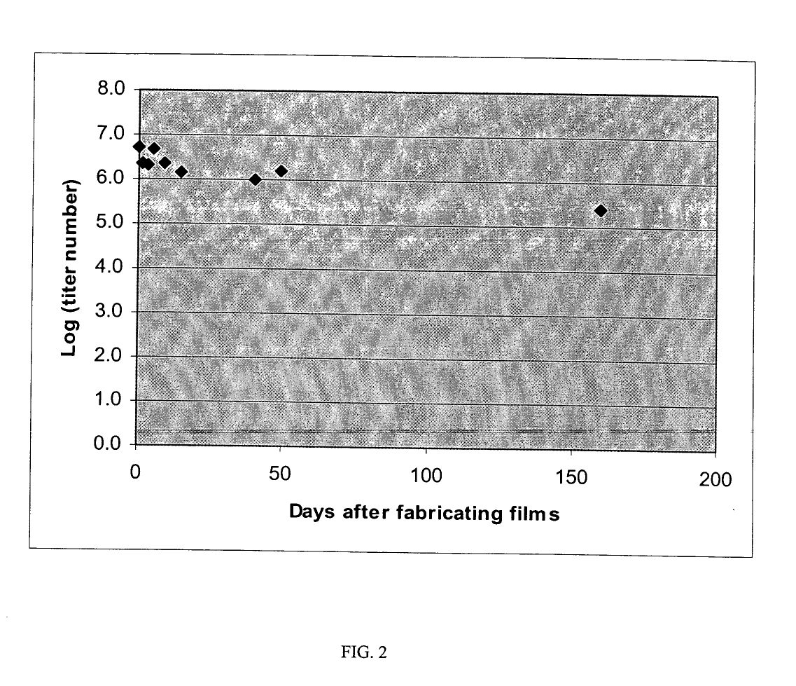

The time-to-infection (time-dependent infecting ability) of the dried phage in film is discussed below. Ten small-size films were fabricated to compare the time dependent titer numbers. In the comparison, 1 μL of the above-described suspension was dried on the sterilized surface of an eppendorff tube in a dessicator for about one day. Titer numbers for each film were measured after suspending each 1 μL film in 1 mL TBS buffer solution (pH 7.5) on a different day over a five-month period. The titer numbers were measured and showed little change for at least seven weeks (FIG. 2).

After five months, the titer number decreased to 10% as compared to the number obtained from a one-day-old film suspension. Elongation and/or optimized infection times may be readily maximized for any biofilm without undue experimentation to those of ordinary skilled in the art.

The biopanning results, including the continued ability of dried phage on film to infect, show that the film fabrication method is a highly efficient storage device of molecular information. For example, the film readily stores high-density engineered DNA and protein information over an extended period of time. In addition, using a bacterial host, the viral components may be replicated easily at any time.

The biofilm may serve to functionalize one or more different types of virus and/or its components and may also be used to express a particular protein or protein unit. The medicinal applications of this technique are extensive as the biofilms can be used in a number of therapeutic avenues including drug discovery, high throughput screening, diagnosis one or more pathologic conditions, and for optimizing disease therapies.

Biopanning for Streptavidin Target. Phage film (FIG. 1A) was fractured at or about a dimension of 1cm ×1 cm and suspended in 1 mL of TBS buffered solution. The suspension (1.1×10⁹ PFU) was exposed to a streptavidin-immobilized Petri plate by the procedure supplied with the Ph.D. 12mer system (New England Biolab). After the second round of biopanning, the randomly selected plaques began to show the sequence pattern, His-Pro-Gln-a specific binding peptide sequence motif for streptavidin (TABLE 2).

TABLE 2

Sequencing results using a streptavidin
target.

| | | |
|---|---|---|
| Sample | Sequence | SEQ ID NO |

| | | |
|---|---|---|
| 2^{nd} Round Sequencing | | |
| LSB-1 | TGHHIHLQAHPI | SEQ ID NO:102 |
| LSB-2 | VPQIPNLISHPM | SEQ ID NO:103 |
| LSB-3 | WELPWIDSNHPQ | SEQ ID NO:104 |
| LSB-4 | IQSTFTLHPWV | SEQ ID NO:105 |
| LSB-5 | KPYLFLQPNYG | SEQ ID NO:106 |
| LSB-6 | NGHVHLPAHPQ | SEQ ID NO:107 |
| LSB-8 | EYTHPLLLAHPI | SEQ ID NO:108 |
| LSB-9 | LPVNAWLVSHPQ | SEQ ID NO:109 |
| LSB-10 | WELPWIDSNHPQ | SEQ ID NO:104 |
| 3rd Round Sequencing | | |
| LSB-11 | WELPWIDSNHPQ | SEQ ID NO:104 |
| LSB-12 | IGSRAETMPWPR | SEQ ID NO:110 |
| LSB-13 | LPVNAWLVSHPQ | SEQ ID NO:109 |
| LSB-14 | QPSWSLLLEHPH | SEQ ID NO:110 |
| LSB-15 | QPSWSLLLEHPR | SEQ ID NO:110 |
| LSB-16 | QPSWSLLLEHPH | SEQ ID NO:110 |
| LSB-18 | WELPWIDSNHPQ | SEQ ID NO:104 |
| LSB-19 | AAKATLSGTASV | SEQ ID NO:111 |
| LSA-1 | VPQIPNWISHPM | SEQ ID NO:103 |
| LSA-2 | WELPWIDSNHPQ | SEQ ID NO:104 |
| LSA-10 | WELPWIDSNHPQ | SEQ ID NO:104 |
| LSC-34 | QDPYSHLLQHPQ | SEQ ID NO:112 |
| 4th Round Sequencing | | |
| LSA-22 | WELPWIDSNHPQ | SEQ ID NO:104 |
| LSA-24 | TTXFPWLQTHPQ | SEQ ID NO:113 |
| LSA-25 | QNWTWSLPHHPQ | SEQ ID NO:114 |
| LSA-26 | WELPWIDSNHPQ | SEQ ID NO:104 |
| LSA-27 | WELPWIDSNHPQ | SEQ ID NO:104 |
| LSA-28 | WELPWIDSNHPQ | SEQ ID NO:104 |
| LSA-29 | WELPWIDSNHPQ | SEQ ID NO:104 |
| LSA-30 | WELPWIDSNHPQ | SEQ ID NO:104 |
| LSC-2 | WELPWIDSNHPQ | SEQ ID NO:104 |
| LSC-5 | WELPWIDSNHPQ | SEQ ID NO:104 |
| LSC-12 | WELPWIDSNHPQ | SEQ ID NO:104 |
| LSC-30 | WELPWIDSNHPQ | SEQ ID NO:104 |

Italicized letters in the sequence represent the streptavidin binding sequence motif.

Time Dependent Infection Ability of Dried Phage in the Film State. 1 μL of the suspension was dried on the sterilized surface of an eppendorff tube in a dessicator. Titer numbers were counted after re-suspending these 1 μL film in 1 mL TBS solution (pH 7.5) on different days for five months (FIG. 2).

The integrity of the dry thin film of phage is extremely high. The thin film stores at least 4×10¹³ phage per square centimeter. Moreover, the number of protein units that may be stored is greater than 7200 times 4×10¹³ phage. As a result, the dry film fabrication method presents an inexpensive and optimal way to store extremely large volumes of biologic material, such as DNA, peptides and proteins, as examples, in a highly organized manner over long periods of time.

As described herein, an engineered viral library may be created, preserved, and reused by fabricating a dry thin film. A genetically engineered M13 phage library was made in a film form from highly concentrated suspension. When the biofilm was suspended again in an appropriate solution, M13 phage remain active and were able to infect a bacterial host. Of importance is that through the use of the present invention, a specific biologic material is preserved, stable, and still active in film form. The biofilm remains stable for more than seven months and retains its activity as shown by its ability to be greater than 95% infectious for at least 5 months.

The biopanning results indicate that most of the 10⁹ phage library information was preserved on the film. In addition, the fabrication of the biofilm is a reversible process with a readily useable application for the storage of high-density engineered molecular information (e.g, DNA, peptide or protein).

With the engineered biofilm of the present invention, three-dimensional memory may be formed that has up to three spatial dimensions. Multiple bit information may be “read” (output) as data that is biologic, optical (such as color wavelengths), magnetic, or electrical depending on the characteristics of the biologic material and or the inorganic compound or nanoparticles in combination with the biologic material. Data is also “written” (input) to the biofilm by creating a chemical, optical, magnetic, or electrical reaction at a specific (e.g., nanoparticle) location. Using the present invention, one or more phage additives (or other biologic materials) may be designed to create a film with very specific binding and or sequence patterns. The resulting film serves as a storage device for input and output of information (as bits of data) with unique optical, electrical, and/or magnetic properties, as further described below. When the biological material is porous, such as in a hydrogel state, for example, reading and writing can be carried out with dissolved labels.

Example of Ordered Biofilm Storage Device with Nanoparticles

Engineered biologic materials such as viruses or bacteriophage (phage) are often able to recognize one or more specific contacting surfaces that help order their appearance on the contacting surface. For bacteriophage, for example, this is through the selection of combinatorial phage display. In this example, the contacting surface recognition results in the ordering of the phage into a self-supporting biofilm that may or may not contain additional inorganic molecules or nanoparticles such as zinc sulfide (ZnS). The presence of the nanoparticles offers additional advantages that help the phage alignment to be magnetically and electrically controlled. This control by an external force does not necessarily require the presence of an additional inorganic molecules; some biologic materials may become ordered externally on the contacting surface without the assistance of an inorganic compound.

Phage recognition of a substrate's contacting surface (e.g., a semiconductor surface) may also be controlled by precoating the substrate with a second biologic material such as a peptide recognition moiety. An example of a precoated substrate is, for example, a semiconductor surface precoated with an additional compound such as indium tin oxide (ITO). This additional compound may or may not be inorganic. For example, some substrates (e.g., glass) may be precoated with an organic compound (e.g., a conducting polymer) to encourage the ordered alignment of the biologic material. Application of an external control, e.g., electric and or magnetic field, may also used to encourage the ordered alignment of biologic material and to create a highly uniform biofilm, where uniformity includes a nonrandom ordering the biologic material on the contacting surface (or substrate). The present invention has been used to demonstrate that such biofilms of the present invention may be stored for more than six months without loss of stability, activity or ability of phage to infect a host. Further examples of the process involved in ordering the biologic material are described below, including examples of methods used to prepare the biologic material.

Phage-display Library. One method of providing a random organic layer is using a Phage-display library, based on a combinatorial library of random peptides containing between 7 and 12 amino acids fused to the pIII coat protein of M13 coliphage, provided different peptides were reacted with crystalline semiconductor structures. Five copies of the pIII coat protein are located on one end of the phage particle, accounting for 10-16 nm of the particle. The phage-display approach provided a physical linkage between the peptide substrate interaction and the DNA that encodes that interaction. The examples described here used as examples, five different single-crystal semiconductors: GaAs(100), GaAs(111)A, GaAs(111)B, InP(100) and Si(100). These substrates allowed for systematic evaluation of the peptide substrate interactions and confirmed the general applicability of the methodology of the present invention for different crystalline structures.

Protein sequences that bond successfully to the specific crystal were eluted from the surface, amplified by, e.g., a million-fold, and reacted against the substrate under more stringent conditions. This binding procedure was repeated five times to select the phage in the library with the most specific binding. After, e.g., the third, fourth and fifth rounds of phage selection, crystal-specific phage were isolated and their DNA sequenced. Peptide binding has been identified that is selective for the crystal composition (for example, binding to GaAs but not to Si) and crystalline face (for example, binding to GaAs(100), but not to GaAs(111)B).

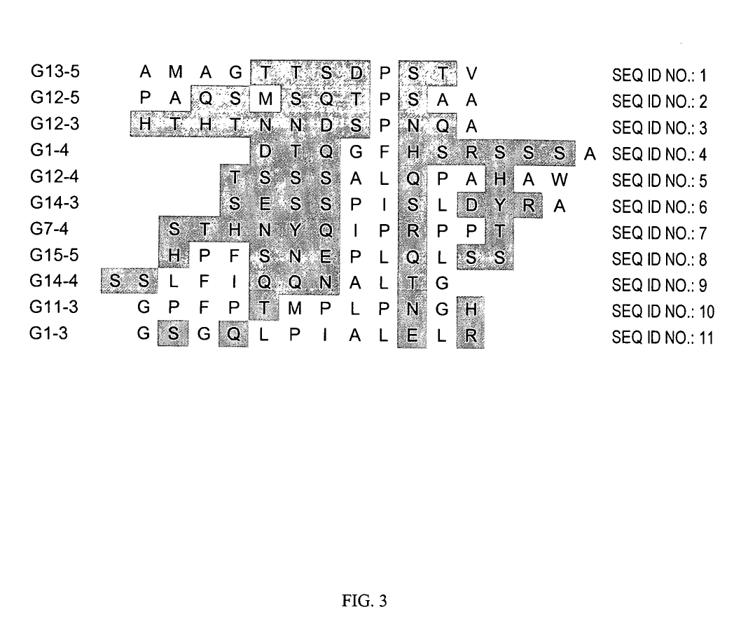

Twenty clones selected from GaAs(100) were analyzed to determine epitope binding domains to the GaAs surface. The partial peptide sequences of the modified pIII or pVIII protein are shown in FIG. 3 (SEQ ID NO: 1-11), revealing similar amino-acid sequences among peptides exposed to GaAs. With increasing number of exposures to a GaAs surface, the number of uncharged polar and Lewis-base functional groups increased. Phage clones from third, fourth and fifth round sequencing contained on average 30%, 40% and 44% polar functional groups, respectively, while the fraction of Lewis-base functional groups increased at the same time from 41% to 48% to 55%. The observed increase in Lewis bases, which should constitute only 34% of the functional groups in random 12-mer peptides from the library used, suggests that interactions between Lewis bases on the peptides and Lewis-acid sites on the GaAs surface may mediate the selective binding exhibited by these clones.

The expected structure of the modified 12-mers selected from the library may be an extended conformation, which seems likely for small peptides, making the peptide much longer than the unit cell (5.65 angstroms) of GaAs. Therefore, only small binding domains would be necessary for the peptide to recognize a GaAs crystal. These short peptide domains, highlighted in FIG. 3, contain serine- and threonine-rich regions in addition to the presence of amine Lewis bases, such as asparagine and glutamine. To determine the exact binding sequence, the surfaces were screened with shorter libraries, including 7-mer and disulphide constrained 7-mer libraries. Using these shorter libraries that reduce the size and flexibility of the binding domain, fewer peptide-surface interactions are allowed, yielding the expected increase in the strength of interactions between generations of selection.

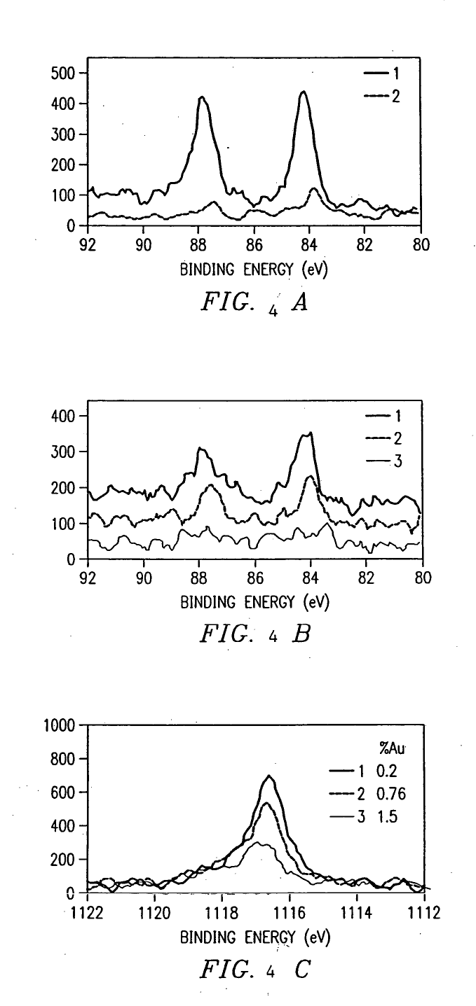

Phage (tagged with streptavidin-labeled 20 nm colloidal gold particles bound to the phage through a biotinylated antibody to the M13 coat protein) were used for quantitative assessment of specific binding. X-ray photoelectron spectroscopy (XPS) elemental composition determination was performed, monitoring the phage substrate interaction through the intensity of the gold 4f-electron signal (FIGS. 4A-C). Without the presence of the G1-3 phage, the antibody and the gold streptavidin did not bind to the GaAs(100)substrate. The gold-streptavidin binding was, therefore, specific to the phage and an indicator of the phage binding to the substrate. Using XPS it was also found that the G1-3 clone isolated from GaAs(100) bound specifically to GaAs(100) but not to Si(100)(see FIG. 4A). In complementary fashion the S1 clone, screened against the (100) Si surface, showed poor binding to the GaAs(100) surface.

Some GaAs clones also bound the surface of InP (100), another zinc-blend structure. The basis of the selective binding, whether it is chemical, structural or electronic, is still under investigation. In addition, the presence of native oxide on the substrate surface may alter the selectivity of peptide binding.

The preferential binding of the G1-3 clone to GaAs(100), over the (1l1)A (gallium terminated) or (111)B (arsenic terminated) face of GaAs was demonstrated (FIGS. 4B and 4C). The G1-3 clone surface concentration was greater on the (100) surface, which was used for its selection, than on the gallium-rich (111)A or arsenic-rich (111)B surfaces. These different surfaces are known to exhibit different chemical reactivities, and it is not surprising that there is selectivity demonstrated in the phage binding to the various crystal faces. Although the bulk termination of both 111 surfaces give the same geometric structure, the differences between having Ga or As atoms outermost in the surface bilayer become more apparent when comparing surface reconstructions. The composition of the oxides of the various GaAs surfaces is also expected to be different, and this in turn may affect the nature of the peptide binding.

The intensity of Ga 2p electrons against the binding energy from substrates that were exposed to the G1-3 phage clone is plotted in FIG. 4C. As expected from the results in FIG. 4B, the Ga 2p intensities observed on the GaAs(100), (111)A and (111)B surfaces are inversely proportional to the gold concentrations. The decrease in Ga 2p intensity on surfaces with higher gold-streptavidin concentrations was due to the increase in surface coverage by the phage. XPS is a surface technique with a sampling depth of approximately 30 angstroms; therefore, as the thickness of the organic layer increases, the signal from the inorganic substrate decreases. This observation was used to confirm that the intensity of gold-streptavidin was indeed due to the presence of phage containing a crystal specific bonding sequence on the surface of GaAs. Binding studies were performed that correlate with the XPS data, where equal numbers of specific phage clones were exposed to various semiconductor substrates with equal surface areas. Wild-type clones (no random peptide insert) did not bind to GaAs (no plaques were detected). For the G1-3 clone, the eluted phage population was 12 times greater from GaAs(100) than from the GaAs(111)A surface.

The G1-3, G12-3 and G7-4 clones bound to GaAs(100) and InP(100) were imaged using atomic force microscopy (AFM). The InP crystal has a zinc-blende structure, isostructural with GaAs, although the In-P bond has greater ionic character than the GaAs bond. The 10-nm width and 900-nm length of the observed phage in AFM matches the dimensions of the M13 phage observed by transmission electron microscopy (TEM), and the gold spheres bound to M13 antibodies were observed bound to the phage (data not shown). The InP surface has a high concentration of phage. These data suggest that many factors are involved in substrate recognition (or recognition of the contacting surface), including atom size, charge, polarity and crystal structure.

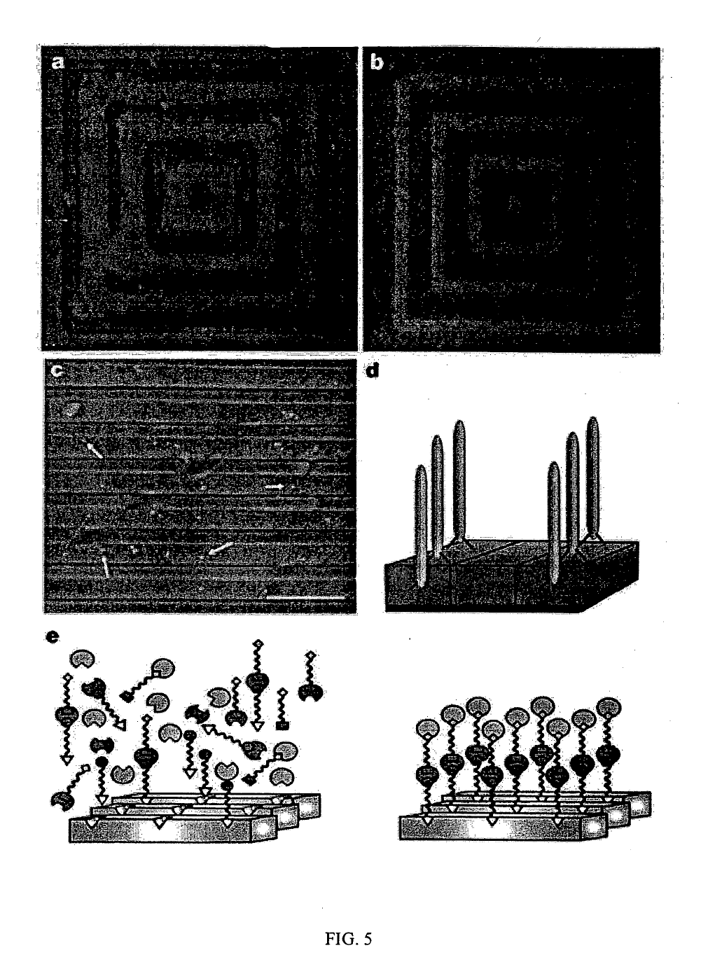

The G1-3 clone (negatively stained) is seen bound to a GaAs crystalline wafer in the TEM image (not shown). The data confirms that binding was directed by the modified pIII protein of G1-3, not through non-specific interactions with the major coat protein. Therefore, peptides of the present invention may be used to direct specific peptide-semiconductor interactions in assembling nanostructures and heterostructures (FIG. 5E).

X-ray fluorescence microscopy was used to demonstrate the preferential attachment of phage to a zinc-blended surface in close proximity to a surface of differing chemical and structural composition. A nested square pattern was etched into a GaAs wafer; this pattern contained 1-pm lines of GaAs, and 4-μm SiO₂ spacing in between each line (FIGS. 5A and 5B) The G12-3 clones were interacted with the GaAs/SiO₂ patterned substrate, washed to reduce non-specific binding, and tagged with an immuno-fluorescent probe, tetramethyl rhodamine (TMR). The tagged phage were found as the three red lines and the center dot, in FIG. 5B, corresponding to G12-3 binding only to GaAs. The SiO₂ regions of the pattern remain unbound by phage and are dark in color. This result was not observed on a control that was not exposed to phage, but was exposed to the primary antibody and TMR (FIG. 5A). The same result was obtained using non-phage bound G12-3 peptide.

The GaAs clone G12-3 was observed to be substrate-specific for GaAs over AlGaAs (FIG. 5C). AlAs and GaAs have essentially identical lattice constraints at room temperature, 5.66 A° and 5.65 A°, respectively, and thus ternary alloys of AlxGal-xAs can be epitaxially grown on GaAs substrates. GaAs and AlGaAs have zinc-blende crystal structures, but the G12-3 clone exhibited selectivity in binding only to GaAs. A multilayer substrate was used, consisting of alternating layers of GaAs and of Al_{0.98}Ga_{0.02}As. The substrate material was cleaved and reacted subsequently with the G12-3 clone.

The G12-3 clones were labeled with 20-nm gold-streptavidin nanoparticles. Examination by scanning electron microscopy (SEM) shows the alternating layers of GaAs and Al_{0.98}Ga_{0.02}As within the heterostructure (FIG. 5C). X-ray elemental analysis of gallium and aluminum was used to map the gold-streptavidin particles exclusively to the GaAs layers of the heterostructure, demonstrating the high degree of binding specificity for chemical composition. In FIG. 5D, a model is depicted for the discrimination of phage for semiconductor heterostructures, as seen in the fluorescence and SEM images (FIGS. 5A-C).

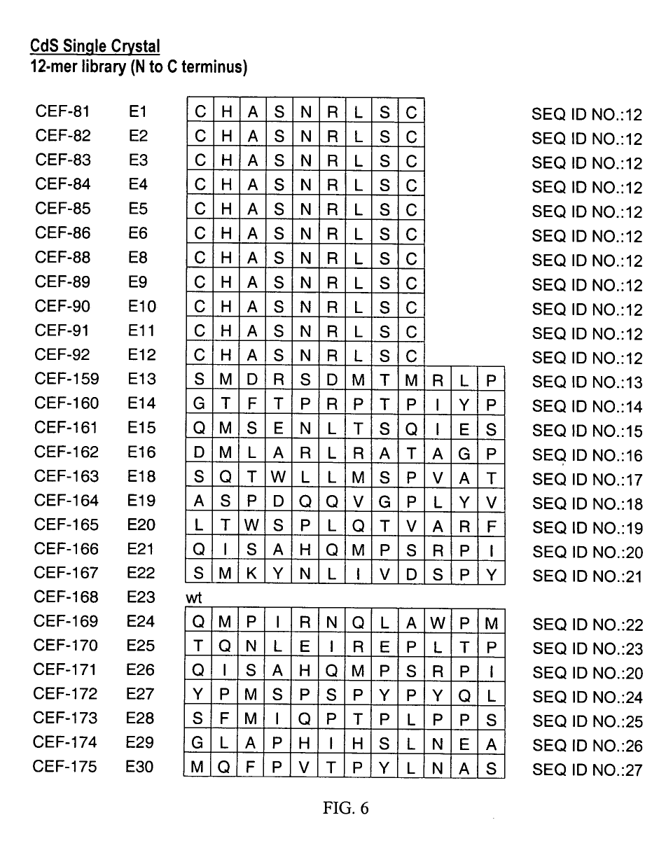

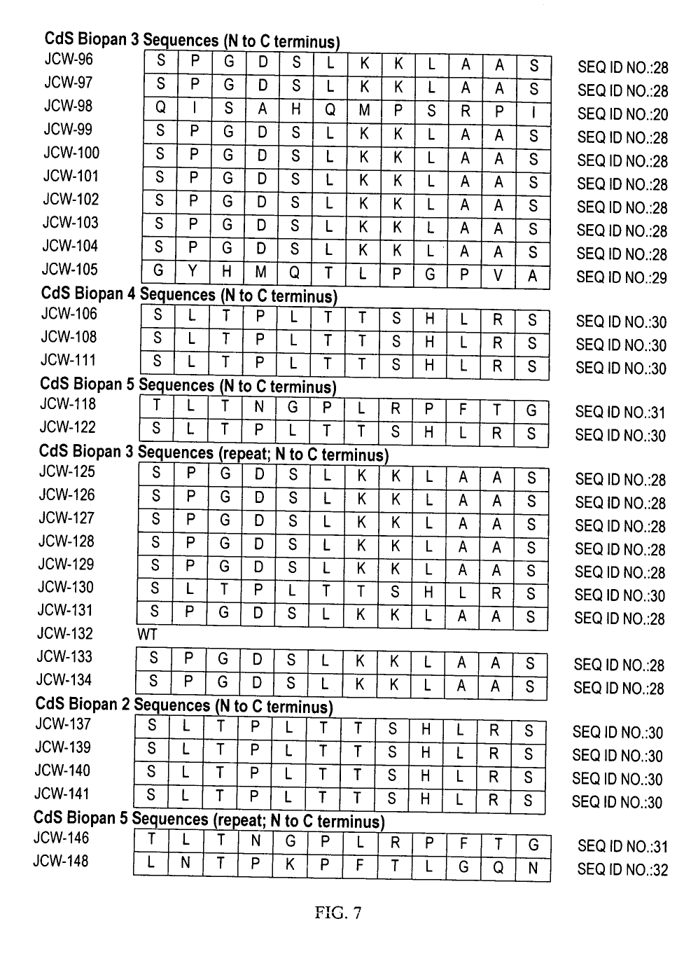

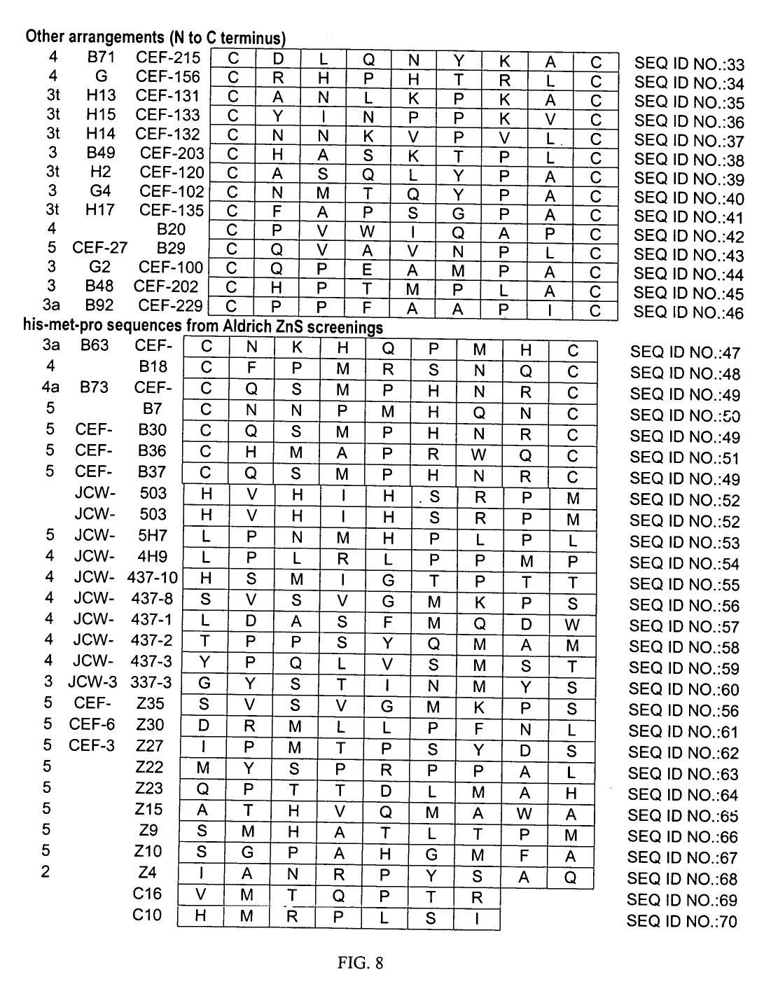

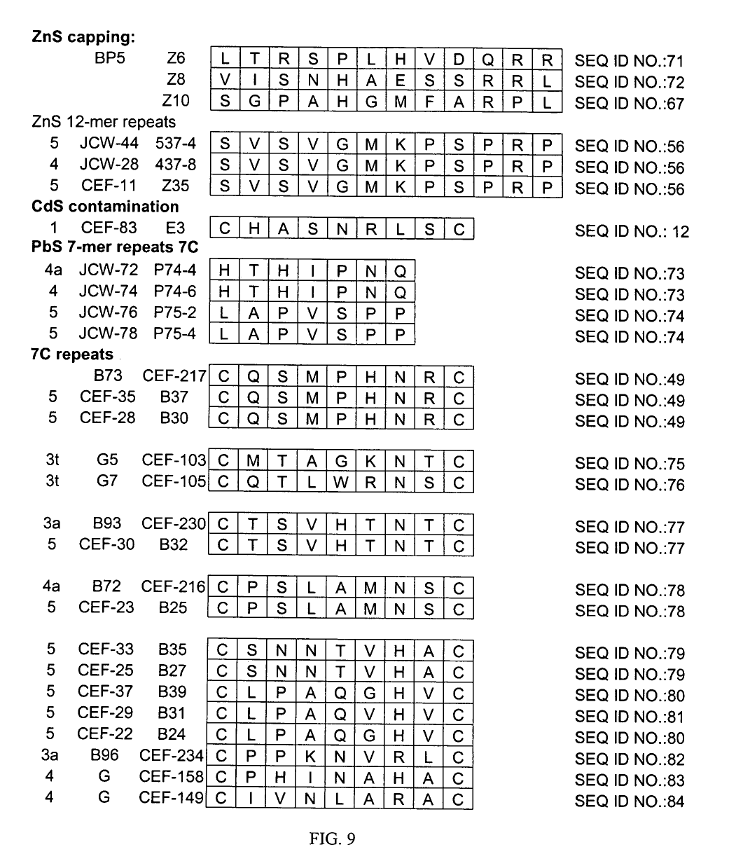

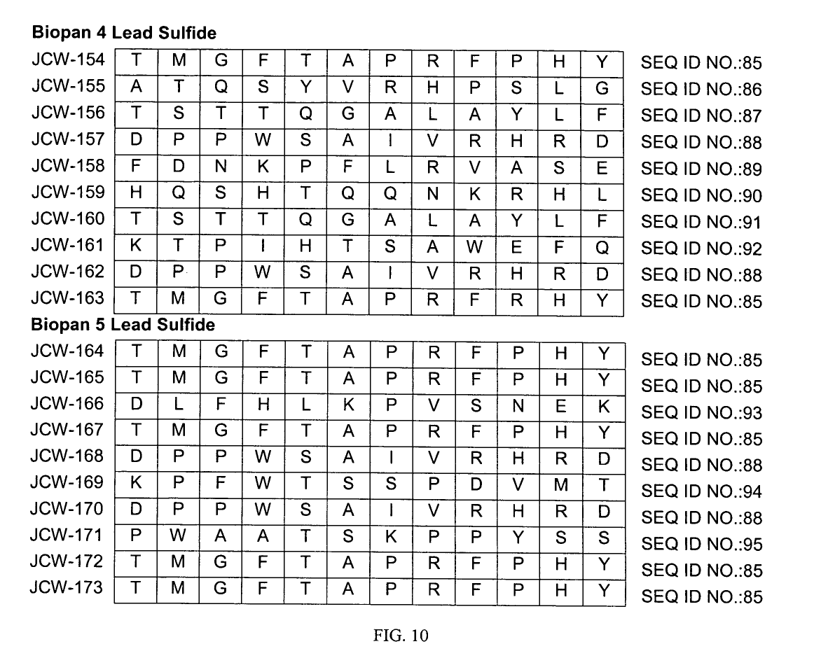

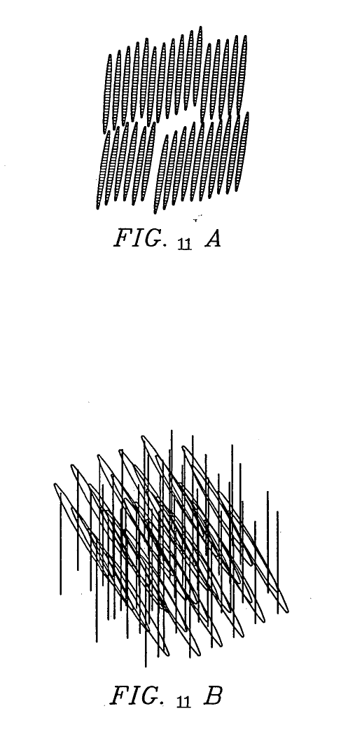

The present invention demonstrates the power use of phage-display libraries to identify, develop and amplify binding between organic peptide sequences and inorganic semiconductor substrates. This peptide recognition and specificity of inorganic crystals has been extended to other substrates, including GaN, ZnS, CdS, Fe₃O₄, Fe₂O3, CdSe, ZnSe and CaCO₃ using peptide libraries. Bivalent synthetic peptides with two-component recognition (FIG. 5E) are currently being designed; such peptides have the potential to direct nanoparticles to specific locations on a semiconductor structure. These organic and inorganic pairs provide powerful building blocks for the fabrication of a new generation of complex, sophisticated electronic structures. Examples of specific amino acid sequences (SEQ ID NOS: 12-95) for peptide recognition of CdS (FIGS. 6-9), ZnS (FIGS. 8, 9), and PbS (FIGS. 9-10) crystals, especially after biopanning, are shown in FIGS. 6-10.

Peptide Creation, Isolation, Selection and Characterization

Peptide Selection. The phage display or peptide library was contacted with the semiconductor, or other, crystals in Tris-buffered saline (TBS) containing 0.1% TWEEN-20, to reduce phage-phage interactions on the surface. After rocking for 1 h at room temperature, the surfaces were washed with 10 exposures to Tris-buffered saline, pH 7.5, and increasing TWEEN-20 concentrations from 0.1% to 0.5%(v/v). The phage were eluted from the surface by the addition of glycine-HCl (pH 2.2) 10 minute, transferred to a fresh tube and then neutralized with Tris-HCl (pH 9.1). The eluted phage were titered and binding efficiency was compared.

The phage eluted after third-round substrate exposure were mixed with their Escherichia coli ER2537 host and plated on LB XGal/IPTG plates. Since the library phage were derived from the vector M13 mp19, which carries the laczα gene, phage plaques were blue in color when plated on media containing Xgal (5-bromo-4-chloro-3-indoyl-β-D-galactoside) and IPTG (isopropyl-β-D-thiogalactoside). Blue/white screening was used to select phage plaques with the random peptide insert. Plaques were picked and DNA sequenced from these plates.

Substrate Preparation. Substrate orientations were confirmed by X-ray diffraction, and native oxides were removed by appropriate chemical specific etching. The following etches were tested on GaAs and InP surfaces: NH₄OH:H₂O (1:10), HCl:H₂O (1:10), H₃PO₄:H₂O₂:H₂O (3:1:50) at 1 minute and 10 minute each time. The best element ratio and least oxide formation (using XPS) for GaAs and InP etched surfaces was achieved using HCl:H₂O for 1 minute followed by a deionized water rinse for 1 minute. An ammonium hydroxide etch was used for GaAs in the initial screening of the library. This etch may also be used for all other GaAs substrate examples, however, those of skill in the art will recognize etches may be used. Si(100) wafers were etched in a solution of HF:H₂O (1:40) for one minute, followed by a deionized water rinse. The surfaces may be taken directly from the rinse solution and immediately introduced to the phage library. Surfaces of control substrates, not exposed to phage, were characterized and mapped for effectiveness of the etching process and morphology of surfaces by AFM and XPS.

Multilayer substrates of GaAs and of Al_{0.98}Ga_{0.02} As were grown by molecular beam epitaxy onto GaAs(100). The epitaxially grown layers were Si-doped (n-type) at a level of 5×10¹⁷ cm⁻³.

Antibody and Gold Labeling. For the XPS, SEM and AFM examples, substrates were exposed to phage for 1 hour in TBS then introduced to an anti-Fd bacteriophage-biotin conjugate, an antibody to the pIII protein of Fd phage, (1:500 in phosphate buffer, Sigma) for 30 minutes and then rinsed in phosphate buffer. A streptavidin-20 nm colloidal gold label (1:200 in phosphate buffered saline (PBS)) was attached to the biotin-conjugated phage through a biotin-streptavidin interaction; the surfaces were exposed to the label for 30 minutes and then rinsed several times with PBS.

X-ray Photoelectron Spectroscopy (XPS). The following controls were done for the XPS examples to ensure that the gold signal seen in XPS was from gold bound to the phage and not non-specific antibody interaction with the GaAs surface. The prepared GaAs(100)surface was exposed to the following: (1) antibody and the streptavidin-gold label without phage, (2) G1-3 phage and streptavidin-gold label without the antibody, and (3) streptavidin-gold label without either G1-3 phage or antibody.

The XPS instrument used was a Physical Electronics Phi ESCA 5700 with an aluminum anode producing monochromatic 1,487-eV X-rays. All samples were introduced to the chamber immediately after gold-tagging the phage (as described above) to limit oxidation of the GaAs surfaces, and then pumped overnight at high vacuum to reduce sample outgassing in the XPS chamber.

Atomic Force Microscopy (AFM). The AFM used was a Digital Instruments Bioscope mounted on a Zeiss Axiovert 100-2 tv, operating in tip scanning mode with a G scanner. The images were taken in air using tapping mode. The AFM probes were etched silicon with 125-mm cantilevers and spring constants of 20±100 Nm⁻¹ driven near their resonant frequency of 200±400 kHz. Scan rates were of the order of 1±5 mms⁻¹. Images were leveled using a first-order plane to remove sample tilt.

Transmission Electron Microscopy (TEM). TEM images were taken using a Philips EM208 at 60 kV. The G1-3 phage (diluted 1:100 in TBS) were incubated with GaAs pieces (500 mm) for 30 minutes, centrifuged to separate particles from unbound phage, rinsed with TBS, and resuspended in TBS. Samples were stained with 2% uranyl acetate.

Scanning Electron Microscopy (SEM). The G12-3 phage (diluted 1:100 in TBS) were incubated with a freshly cleaved hetero-structure surface for 30 minutes and rinsed with TBS. The G12-3 phage were tagged with 20 nm colloidal gold. SEM and elemental mapping images were collected using the Norian detection system mounted on a Hitachi 4700 field emission scanning electron microscope at 5 kV.

Fabrication of Ordered Hybrid Biofilm Storage Devices

The present inventors have recognized that organic-inorganic hybrid materials (those materials that include both organic and inorganic compounds) offer new routes for novel material development. Size controlled structures in the nanoscale range (nanostructures) are especially useful in microeletronics and offer optical, magnetic, and electric-tunable properties to materials such as semiconductors. The biologic material with its organic component may further modify the inorganic morphology, phase, and nucleation direction of the structure, especially at the nanoscale level. This hybrid creates a highly unique microenvironment with location-specific information or data. The ability to store this information for extended lengths of time is critical to its success as a storage tool for information processing, gathering and analysis.

Using phage as an example, it is clear that a biologic material with its generally monodispersed nature offers the new material a unique set of new criteria in which to store variable pieces of information. With the present invention, highly ordered structures with ordering on the nanometer scale were composed. Multi-length scale alignment of II-VI semiconductor material using genetically engineered, self-assembling, biological molecules, (e.g., M13 bacteriophage that have a recognition moiety of specific semiconductor surfaces) create optimal devices for long-term data storage. Thus, the monodisperse biomaterials having anisotrophic shapes are an alternative way to build well-ordered structures. Nano- and multi-length scale alignment of II-VI semiconductor material was accomplished using genetically engineered M13 bacteriophage that possess a recognition moiety (a peptide or amino acid oligomer) for specific semiconductor surfaces.

Fd virus smectic ordering structures that have both a positional and directional order have been characterized. The smectic structure of Fd virus has potential application in both multi-scale and nanoscale ordering of structures to build 2-dimensional (2D) and 3-dimensional (3D) alignment of particles in the nanometer scale (herein referred to as nanoparticles). Bacteriophage M13 was used because it can be genetically modified, has been successfully selected to have a shape identical to the Fd virus, and has specific binding affinities for II-VI semiconductor surfaces. Therefore, M13 is an ideal source for smectic structure that can serve in multi-scale and nanoscale ordering of nanoparticles.

The present inventors have used combinatorial screening methods to find M13 bacteriophage containing peptide “inserts” that are capable of binding to semiconductor surfaces. These semiconductor surfaces included materials such as zinc sulfide, cadmium sulfide and iron sulfide. Using the techniques of molecular biology known to those of ordinary skill in the art, a biologic material such as bacteriophage combinatorial library clones that bind specific semiconductor material surfaces, are used. In general, biologic material is one that is readily available in large quantity or may be amplified readily for large-scale manufacturing. The phage is amplified cloned and amplified up to concentrations high enough for liquid crystal formation.

The anisotrophic shape of bacteriophage was exploited as a method to build well-ordered nanoparticle layers by use of biological selectivity and self-assembly. For example, the filamentous bacteriophage, Fd, has a long rod shape (length: 880 nm; diameter: 6.6 nm) and monodisperse molecular weight (molecular weight: 1.64×10⁷) that results in the bacteriophage's lyotropic liquid crystalline behavior in highly concentrated solutions. In the present invention, M13, a similar filamentous bacteriophage, was genetically modified to bind nanoparticles such as zinc sulfide, cadmium sulfide and iron sulfide. The monodisperse bacteriophage, M13, was prepared through standard amplification methods.

Nano- and Mesoscale Ordering. The ordering of bacteriophage on the nano- and mesoscale level shows that the biologic material may form nanoscale arrays of nanoparticles. These nanoparticles are further organized into micron domains and into centimeter length scales. The semiconductor nanoparticles show quantum confinement effects, and can be synthesized and ordered within the liquid crystal.

Genetically engineered M13 bacteriophage that have specific binding properties to semiconductor surfaces were amplified and purified using standard molecular biological techniques. 3.2 mL of bacteriophage suspension (concentration: ˜10⁷ phages/μL) and 4 mL of overnight culture were added to 400 mL LB medium for mass amplification. After amplification, ˜30 mg of pellet was precipitated. The suspensions were prepared by adding Na₂S solutions to ZnCl₂ doped A7 phage suspensions at room temperature. The highest concentration of A7-phage suspension was prepared by adding 20 μL of 1 mM ZnCl₂ and Na₂S solutions, respectively into the ˜30 mg of phage pellet. The concentration was measured using extinction coefficient of 3.84 mg/mL at 269 nm.

As the concentration of the isotropic suspension is increased, nemetic phase that has directional order, cholesteric phase that has twisted nemetic structure, and smectic phase that has directional and positional orders as well, are observed. These phases had been observed in Fd viruses that did not have nanoparticles. Bacteriophage M13 suspension containing specific peptide inserts were made and characterized. Uniform 2D and 3D ordering of nanoparticles was observed throughout the samples.

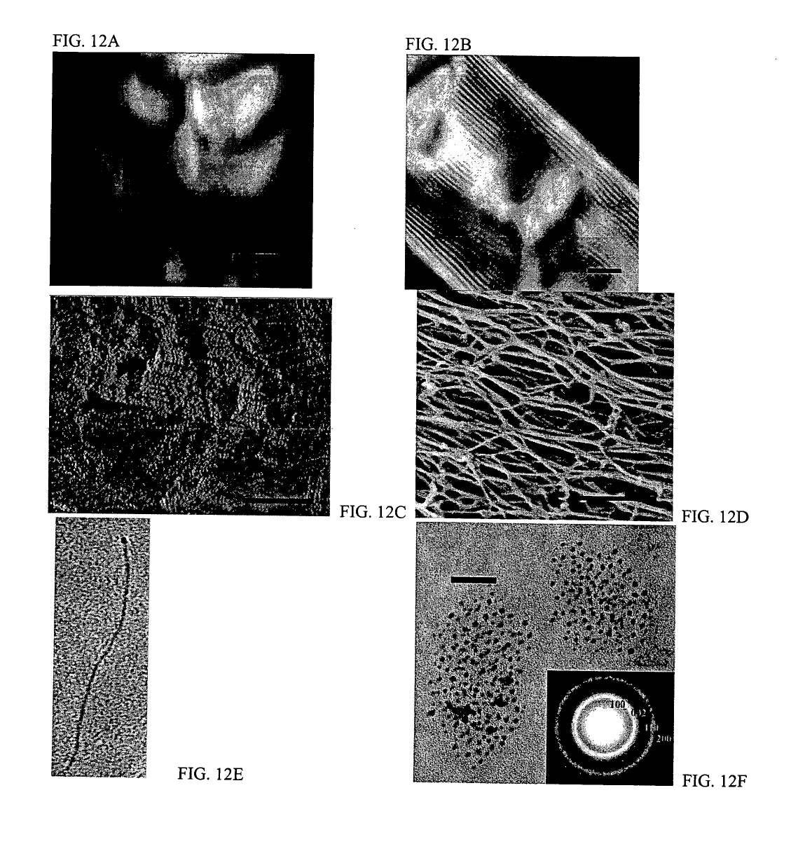

Atomic Force Microscopy (AFM). The AFM used is the same as previously described. FIGS. 11A and 11B are schematic diagrams of the smectic alignment of M13 phages observed using AFM. Additionally, 5 μL of M13 suspension (concentration: 30 mg/mL) of M13 bacteriophage suspension was dried for 24 hours on the 8 mm×8 mm mica substrate that was silated by 3-amino propyl triethyl silane for 4 hours in the dessicator. Images were taken in air using tapping mode. Self-assembled ordering structures were observed due to the anisotropic shape of M13 bacteriophage, 880 nm in length and 6.6 nm in width. In FIG. 12C, M13 phage lie in the plane of the photo and form smectic alignment.

Transmission Electron Microscopy (TEM). TEM images were taken as described previously.

Scanning Electron Microscopy (SEM). Preparation of samples and use of SEM is as previously described. The critical point drying samples of bacteriophage and ZnS nanoparticles smectic suspension (concentration of bacteriophage suspension 127 mg/mL) were prepared. In FIG. 12D, nanoparticles rich areas and bacteriophage rich areas were observed. The length of the separation between nanoparticles and bacteriophage correspond to the length of bacteriophage. The ZnS wurzite crystal structure was confirmed by electron diffraction pattern using dilution sample of the smectic suspension with TEM.

Polarized Optical Microscopy (POM). M13 phage suspensions were characterized by POM. Each suspension was filled to glass capillary tube of 0.7 mm diameter. The highly concentrated suspension (127 mg/mL) exhibited iridescent color [5] under the paralleled polarized light and showed smectic texture under the cross-polarized light as FIG. 12A. The cholesteric pitches, FIG. 12B can be controlled by varying the concentration of suspension as shown in TABLE 3. The pitch length was measured and the micrographs were taken 24 hours later from the preparation of samples.

TABLE 3
Cholesteric Pitch and Concentration Relationship.

| Concentration | Pitch length |
|---|---|
| (mg/mL) | (μm) |
| 76.30 | 31.9 |
| 71.22 | 51.6 |
| 56.38 | 84.8 |
| 50.52 | 101.9 |
| 43.16 | 163.7 |
| 37.04 | 176.1 |
| 27.54 | 259.7 |

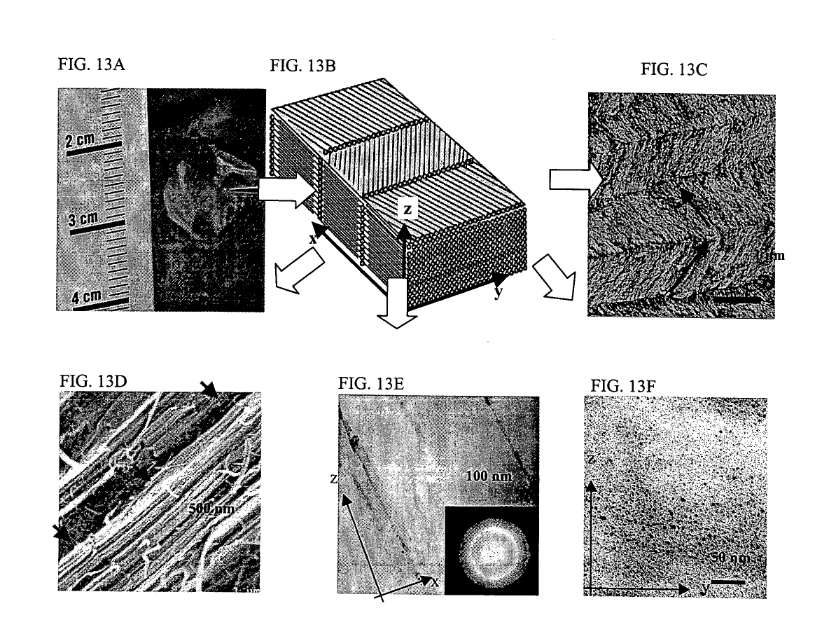

Preparation of the Nanocrystal Biofilm. Bacteriophage pellets were suspended with 400 μL of Tris-buffered saline (TBS, pH 7.5) and 200 μL of 1 mM ZnCl₂ to which 1 mM Na₂S was added. After rocking for 24 hours at room temperature, the suspension that was contained in a 1 mL eppendorff tube, was dried slowly in a dessicator for one week. A semi-transparent film ˜15 μm thick was formed on the inside of the tube. This film, FIG. 13A, was carefully taken using a tweezers.

SEM Observation of Nanocrystal Biofilm. Nanoscale bacteriophage alignment of the A7-ZnS film were observed using SEM. In order to carry out SEM analysis the film was cut then coated via vacuum deposition with 2 nm of chromium in an argon atmosphere. Highly close-packed structures were observed throughout the sample (see FIG. 13D). The average length of individual phage, 895 nm is reasonable analogous to that of phage, 880 nm. The film showed the smectic like A or C like lamellar morphologies that exhibited periodicity between the nanoparticle and bacteriophage layers. The length of periodicity corresponded to that of the bacteriophage. The average size of nanoparticle is ˜20 nm analogous to the TEM observation of individual particles.

TEM Observation of Nanocrystal Biofilm. ZnS nanoparticle alignment was investigated using TEM. The film was embedded in epoxy resin (LR white) for one day and polymerized by adding 10 uL of accelerator. After curing, the resin was thin sectioned using a Leica Ultramicrotome. These ˜50 nm sections were floated on distilled water, and picked up on blank gold grids. Parallel-aligned nanoparticles in a low, which corresponded to x-z plane in the schematic diagram, were observed, FIG. 13E. Since each bacteriophage had 5 copies of the A7 moieties, each A7 recognize one nanoparticle (2˜3 nm size) and aligned approximately 20 nm in a width and extended to more than two micrometers in length. The two micrometers by 20 nm bands formed in parallel each band separated by ˜700 nm. This discrepancy may come from the tilted smectic alignment of the phage layers with respect to observation in the TEM. A y-z axis like nanoparticle layer plane was also observed like FIG. 5F. The SAED patterns of the aligned particles showed that the ZnS particles have the wurzite hexagonal structure.

AFM Observation of Nanocrystal Biofilm. The surface orientation of the viral film was investigated using AFM. In FIG. 5C, the phage were shown to have formed an parallel aligned herringbone pattern that have almost right angle between the adjacent director normal (bacteriophage axis) on most of surface that is named as smectic O. The film showed long range ordering of normal director that is persistent to the tens of micrometers. In some of areas where two domain layers meet each other, two or three multi-length scale of bacteriophage aligned paralleled and persistent to the smectic C ordering structure.

Nano and multi-length scale alignment of semiconductor materials using the recognition and self-ordering method and the composition of the present invention enhances the future microfabrication of electronic devices. These devices have the potential to surpass current photolithographic capabilities. Other potential applications of these materials include optoelectronic devices such as light-emitting displays, optical detectors, and lasers, fast interconnects, nano-meter scale computer component and biological sensors.

Stabilizing a Biofilm Storage Device and Maintaining Biologic Activity

The biofilm storage device of the present invention may be used to store biologic (e.g., organic) materials such as enzymes and antibodies. In one embodiment of the present invention, biologic molecules such as enzymes that retain their biologic activity are stored as a biofilm. The activity is readily monitored over time based on the known properties of the enzyme. In one embodiment, β-galactosidase, a reporter enzyme, is prepared in a biofilm and found to retain long-term enzyme stability and activity.

In another embodiment of the present invention, storage solutions (e.g., sucrose) are used to enhance the stability and long-term activity of the biologic material (e.g., enzyme). Furthermore, the present example illustrates that addition of a storage solution used as a stabilizer will enhance the preservation of a biofilm storage device, and may be especially important when biologic activity is a key component of the biofilm.

In order to visualize the structure and function of a biologic material used as a biofilm storage device, light properties of the biologic material or light-emitting molecules that attach to the biologic material may be monitored. For example, a green fluorescent protein variant (GFPuv) that emits green light at a maximum emission wavelength of 509 nm may be used to attach to the biologic material (e.g., enzyme or antibody). Furthermore, the light emitting properties may be imaged using instruments well known to one of ordinary skill in the art of biologic imaging. One example of an imaging instrument is confocal microscopy.

In another embodiment of the present invention, a biologic material used as a storage device is allowed to contact another biologic material. Either biologic material may be modified in whole or in part to customize the biofilm as needed. For example, biofilms including a biologic material such as bacteriophage, may be modified by changing the proteins displayed at the biologic material (e.g., baceriophage) surface or by targeting peptides that specifically attach to the biologic material and may also attach to another target (e.g., biologic material such as protein, antibody, drug, or nucleic acid) or other stabilizer that result in enhanced stability of the biofilm storage device.

Storage temperature can be, for example, about room temperature. Storage temperature can be, for example, about 10° C. to about 40° C., and more particularly, about 20° C. to about 30° C. These storage temperatures can be maintained for any length of time including at least 7 weeks, at least 5 months, at least 6 months, or at least 7 months.

Preparing a stable enzyme-containing biofuim storage device. The enzyme β-galactosidase in phosphate buffered saline (PBS) solution (pH 7.0) was mixed with stock solutions of glucose, sucrose, and M13 phage to obtain concentrations of 0.5 mg/mL β-galactosidase, 5 mg/mL glucose, 50 mg/mL sucrose, and 1.25 mg/mL phage. Aliquots (20 μL) of the solution were placed in 1.5 mL Eppendorf tubes, dried in a dessicator for two days, and stored at room temperature. The dried viral films were suspended in 500 μL of PBS solution (pH 7.0). 100 μL of the suspension and 700 μL of o-nitrophenyl galactoside (ONPG) (1.5×10⁻² M) were combined in a disposable cuvette. The enzyme activities (units) were obtained by monitoring an increase of absorbance of o-nitrophenol (ONP) at 420 nm for 10 minutes with 30 seconds interval. One unit of activity was defined as the amount of enzyme that can catalyze the transformation of 1 μmol of ONPG into ONP in 1 minute at 25 degrees Centrigrade (pH 7.0).

Monitoring biologic activity and stability in a biofilm. A DNA-encoding GFPuv (Clontech) was amplified by PCR and subcloned into pFLAG-CTC vector (Sigma) for the expression of GFPuv-FLAG in Excherichia coli. Whole cell extract was prepared, and the expressed GFPuv-FLAG was purified using anti-FLAG M2 affinity gel column (Sigma). The mixture of GFPuv-FLAG, phage, and glucose:sucrose (1:10 w/w) was prepared with the final concentrations as: 100 μg/mL GFPuv-FLAG, 5 mg/mL phage, 5 mg/mL glucose, and 50 mg/mL sucrose. At least about 10 μL of the mixture was dispensed on a glass slide and dried in a desiccator for a day. GFPuv-FLAG stability was monitored using confocal fluorescence microscopy. Concentrations of glucose:sucrose were 2.5 and 25 mg/mL.

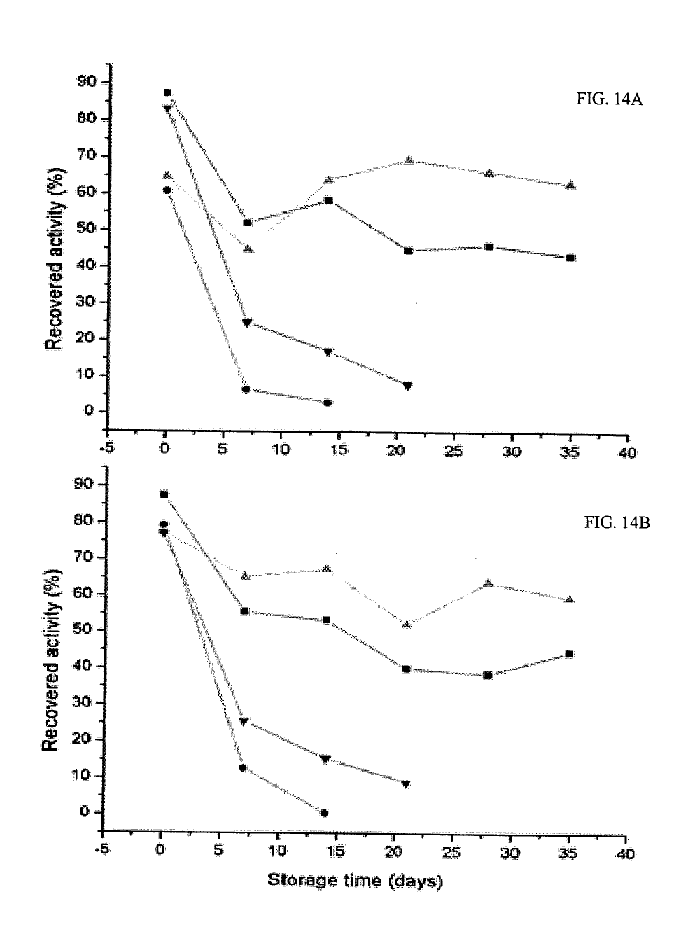

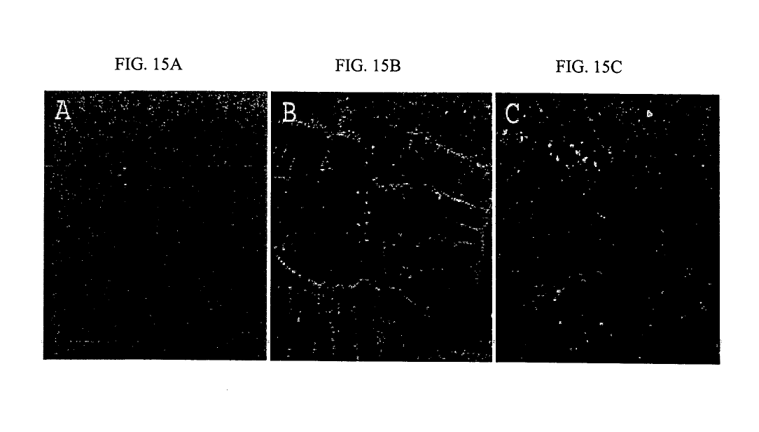

After storage of the prepared biofilm storage device, the measured activity of β-galactosidase was found to be improved with the addition of glucose:sucrose as a storage solution or stabilizer (FIGS. 14A and 14B). Samples used as controls were those biologic materials (e.g., β-galactosidase) prepared as described above in the absence of bacteriophage and sugar and dried in a desiccator. Clearly storage of the enzyme as a biofilm storage device did not affect enzyme activity. Biofilm storage devices containing β-galactosidase and stored after freeze-drying or air-drying showed similar enzyme activity. Interestingly, enzyme activity was also improved in the presence of another biologic materials (e.g., bacteriophage) as well as in the presence of a stabilizer (i.e., storage solution).

FIG. 15 illustrates the confocal microscopy images with GFPuv after excitation at 361 nm. The images illustrate that the addition of a stabilizer such as a glucose:sucrose storage solution improves the biofilm surface and prevents potential deformation of the biologic material during the fabrication (preparation) process. FIG. 15A shows a strong GFPuv signal and homogenous biofilm surface. In the absence a glucose:sucrose storage solution, the film exhibits numerous deformations at the film surface (FIGS. 15B and 15C).

In addition, when the biological material comprises multiple display sites, the biological material can be genetically engineered so that one or more of these display sites is modified. For example, the M13 bacteriophage can be modified at the pIII, P7, p8, or p9 sites to include specific binding peptides. For example, one end of a biological material can be modified to bind specifically to a surface, and the other end of the biological molecule can be modified to bind to a component which is being stored with a goal of stable storage such as a vaccine or a functional protein.

The present invention is thus able to store biologically active biologic materials with activity that persists throughout the storage interval. With additional modifications, biologic and/or other active properties of the biofilm (e.g., electrical, magnetic, optic, mechanical) may be readily manipulated as needed. Activity can be further modified without undue experimentation by changing the biologic surface via altering surface binding properties, through the addition of storage stabilizers and or inhibitors, and by the addition of other organic or inorganic molecules. Storage solutions that stabilize the biologic material include sugar-containing solutions such as glucose, sucrose, glycol, glycerol, polyethylene glycol.

The present invention improves biofilm technology by fabricating stable films composed of biologic materials (including one or more organic and or inorganic materials) that may undergo long-term storage while retaining the original information and/or activity. Engineered materials may be used to fabricate ordered films (biofilms) with long-term activity and stability that hold and store information that is biologic, electric, magnetic, and/or optical. More importantly, the information may be tailored and of extremely high density, thereby serving as an efficient and cost-effective method of storing nanoscale data. The use of these biofilms extends into applications such as medicine, electronics, computer technology and optics, as examples.

Using the compositions and methods of the present invention nano- and multi-length scale alignment of semiconductor materials was achieved using the recognition and self-ordering system described herein. The recognition and self-ordering of semiconductors may be used to enhance micro fabrication of electronic devices that surpass current photolithographic capabilities. Application of these materials include optoelectronic devices such as light emitting displays; optical detectors and lasers; fast interconnects; and nano-meter scale computer components and biological sensors. Other uses of the biofilms created using the present invention include well-ordered liquid crystal displays, organic-inorganic display technology, and films for high-throughput processing, screening and drug discovery, devices for diagnosis, medical testing and analysis; implant surfaces for data storage and specific data recognition, as examples.

The films, fibers and other structures developed from the biofilm of the present invention may even include high-density sensors for detection of small molecules including biological toxins. Other uses include optical coatings and optical switches. Optionally, scaffoldings for medical implants or even bone implants; may be constructed using one or more of the materials disclosed herein, in single or multiple layers or even in striations or combinations of any of these, as will be apparent to those of skill in the art. Other uses for the present invention include electrical and magnetic interfaces, or even the organization of 3D electronic nanostructures for high-density storage, e.g., for use in quantum computing. Alternatively, variable-density and stable storage of viruses for medical application that can be reconstituted, e.g., biologically compatible vaccines, adjuvants and vaccine containers may be created with the films and or matrices created with the present invention.

Information storage based on quantum dot patterns for identification, e.g., department of defense friend or foe identification, may be incorporated in fabric of armor or coding. The present biofilms may even be used to code and identify money.

Other applications include drug delivery, including systems such as, for example, Depomed with layered film assemblies in drug capsules; medical device coatings; and controlled release applications such as, for example, breath mints.

Additional description and working examples are provided below for Embodiment A and Embodiment B. Embodiment A includes a set of cited references, and embodiment B includes a set of cited references.

### ADDITIONAL DESCRIPTION AND WORKING EXAMPLES (EMBODIMENT A)

The paper by Lee et al. “Chiral Smectic C Structures of Virus-Based Films” Langmuir, 2003, 19, 1592-1598 is hereby incorporated by reference in its entirety including abstract, figures, tables, introduction, experimental section, references cited, and results and discussion section.

Additional materials were prepared which can be used as films in storage applications, as well as other applications. In these additional experiments, long-range ordered virus based films were fabricated using M13 phage (viruses) which were aligned and assembled using the meniscus phenomena. Their ordered structures and morphologies were studied and characterized using polarized optical microscopy (POM), atomic force microscopy (AFM) and scanning electron microscopy (SEM). M13 virus particles which are 880 nm in length were the basic building block of the fabricated films. Due to the unique micrometer length scale of viruses, the smectic ordering of microscopy techniques and compared with a theoretical model of chiral liquid crystal structures. From the results of POM, AFM and SEM, the viral films were determined to have a chiral smectic C structure. By comparing ordering of film formation as a function of virus concentration and the formation of bundle-like domain structure found in viral thin films, a mechanism of film formation can be suggested. These virus based film structures are organized on multiple length scales, easily fabricated, and allow integration of aligned semiconductor and magnetic nanocrystals. These self-assembled hybrid materials can be used in, for example, in miniaturized self-assembled electronic devices.

Building well ordered and defect-free two- and three-dimensional structures on the nanometer scale has become a critical issue for the construction of next-generation optical, electronic and magnetic materials and devices.¹⁻⁵ Although numerous techniques to organize nanoparticles and other nanometer-sized objects at small-length scales have been attempted, including traditional hydrogen bonding recognition to newly developed DNA linker system, extending such patterns to the micrometer scale has proven difficult. ⁶⁻⁷ The use of biological materials can provide alternative routes to conventional processing methods for the construction of miniaturized nanoscale devices.^{5,8} Several desirable features of biological systems include the ability to orchestrate precise self assembling structures, highly evolved molecular recognition for both organic and inorganic materials and ability to synthesis inorganic materials into hierarchical structures. Several types of biomaterials have been exploited in the nanoscale assembly of complex architectures.^{5,8-13} Recently, a new method for self-assembling quantum dots in well ordered nanocrystal films has been reported using nanocrystal-functionalized M13 phage.⁵ M13 viruses were genetically engineered to nucleate or bind desired materials on one-end of the M13 virus. These nanocrystal-functionalized viral liquid crystalline building blocks were grown into hybrid ordered self-supporting films. The resulting nanocrystal hybrid films were ordered at the nanometer scale and at the micrometer scale into 72 μm periodic patterns. The smectic O structures on the surfaces and smectic A or C structures in the bulk of the nanocrystal hybrid film were reported.

Here, more extensive characterization of these virus based films including chiral effects of virus building blocks are reported and provide strong evidence that these virus based films are organized into chiral smectic C structures. The viral films fabricated from different concentrations the films. The viral film results are compared with the ZnS nanocrystal hybrid viral film previously reported.

This represents a novel example of a long range ordered lyotropic liquid crystalline chiral smectic C film. This is further evidence that support Meyer's theoretical suggestion that smectic C structures formed from the chiral molecules should have chiral smectic C structures.¹⁴ Although several microscopy techniques have been used to visualize ordered liquid crystalline materials, understanding of molecular orientation of the liquid crystalline ordered structure has been generally limited by the small size and softness of the mesogen units of conventional liquid crystalline materials.^{15,16,30,34} However, using micrometer scale biomolecules (viruses), surface defects of chiral smectic C structures were easily characterized. Moreover, in order to fabricate defect free and well-ordered complex architectures using virus building block, a basic understanding of the surface and bulk structures of these materials is important for further application of the semiconductor nanocrystal hybrid virus films.

TABLE 1
Thickness of the viral films as a function of the initial bulk concentration.

| | Sample number | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| conc. (mg/ml) | 9.93 | 9.70 | 8.63 | 7.60 | 6.88 | 6.38 | 5.09 | 4.39 | 3.36 | 2.59 | 1.79 | 1.05 |
| Thickness (μm) | 12.9 | 12.8 | 7.55 | 6.11 | 5.29 | 6.53 | 4.34 | 3.45 | 2.16 | 2.91 | 1.60 | N/A |

TABLE 2
A. Chiral smectic C pitches measured by polarized
optical microscopy (POM) and laser light diffraction.

| | Sample number | | | | | | |
|---|---|---|---|---|---|---|---|
| | 1 | 2 | 3 | 4 | 5 | 6 | 7 |
| POM (μm) | 36.79 | 31.63 | 30.30 | 27.37 | 36.46 | 41.03 | 41.04 |
| Laser (μm) | 35.76 | 32.34 | 31.54 | 29.28 | 35.41 | 42.05 | N/A |

B. Periodic zig-zag smectic A patterns measured by POM.

| | Sample number | | | |
|---|---|---|---|---|
| | 7 | 8 | 9 | 10 |
| POM (μm) | 97.43 | 93.87 | N/A | 62.38 |

Experimental (Embodiment A):

Viral Film Preparation:

M13 phage were prepared using standard biological methods of amplification and purification described previously.⁵ Twelve different concentrations of M13 phage (800 μl each) were prepared as shown in table 1. After transferring to ependorff tubes (1 cm in diameter and 4 cm in length), the suspensions were allowed to dry in a dessicator for three weeks (weight loss in the drying process: ˜100 mg per day). Cast films were formed on the wall of the ependorff tubes as the solvent evaporated.

Polarized Optical Microscopy:

POM images were obtained using Olympus polarized optical microscope. Micrographs were taken using SPOT Digital camera (Diagnostic Inc.). The optical activity was also observed by changing the angles between the polarizer and analyzer. The polarized optical microscope was used to measure the chiral smectic C spacing patterns.

Scanning Electron Microscopy:

A scanning electron microscope (LE01530) was used to observe the surface morphologies of the viral films. In order to enhance the contrast and to avoid surface charging effects under the electron beam, the viral films were coated with chromium using a plasma ion beam sputtering machine. In order to measure the thickness of the film sample, the sample holder was tilted ˜80 degrees from the horizontal plane and mounted to the SEM sample stage.

Atomic Force Microscope:

Atomic force microscope (AFM) (Digital Instruments) was used to study the surface morphologies of the viral film. The images were taken in air using tapping mode. The AFM probes were etched silicon with 125 μm cantilevers and spring constants of 20-100 N/m driven near their resonant frequency of 250-350 kHz. Scan rates were of the order of 1-40 μm/s.

Laser Light Diffraction:

Laser beam diffraction (He-Ni laser:632.8 nm) was used to measure a chiral smectic C pitch of the viral film. The distance between the screen and sample was 200 cm. The diffraction pattern was recorded by Sony Mavica digital camera. Spacing was calculated by measuring the first order Bragg diffraction spot.

Film Formation and Thickness

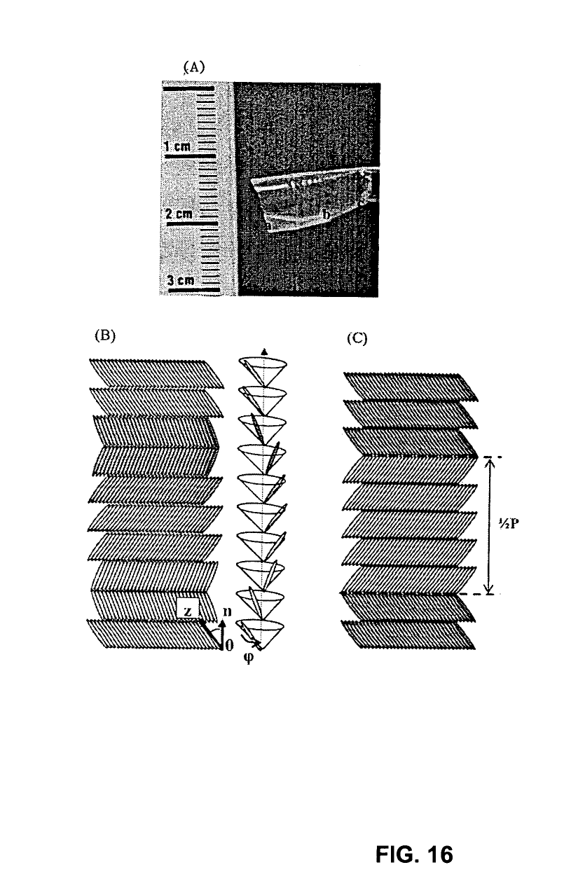

The cast films fabricated from the initial virus concentration between 1.79-9.93 mg/ml were self-supporting and could be manipulated with forceps (FIG. 16A). Under these conditions and for this viral material, viral films fabricated from concentrations under ˜1 mg/ml generally were too thin to be self-supporting when removed from the substrates. The film thickness was measured using SEM and showed in table 1. Generally, the thickness was proportional to the initial concentration of the bulk suspension.

Chiral Smectic C Ordered Films:

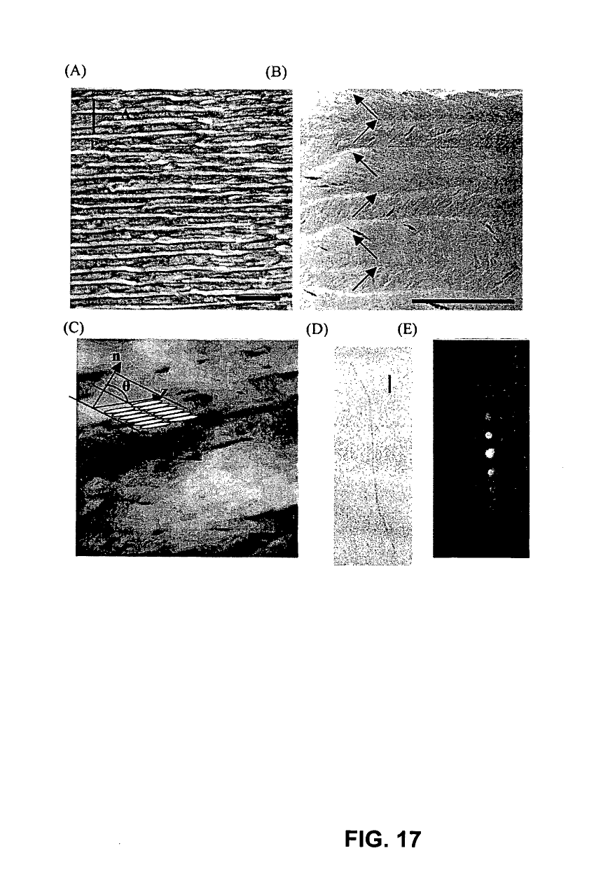

POM images of the viral film formed from the initial concentration 9.93 mg/ml (sample 1) revealed optically active dark and bright band patterns (FIG. 17A). Periodic spacing of these patterns was 36.79±0.95 μm and the patterns were continued over the centimeter-scale. Using optical microscopy, when the focus level through the optic axis was changed at higher magnification, parallel band patterns smaller than 1 μm were also observed. These fine band patterns corresponded to the smectic layer structure of M13 virus molecules. The film was determined to be optically active as evident by the change in intensity of the alternating dark and bright band pattern as the angles between the polarizers were rotated.

These optically active dark and bright band patterns are consistent with a chiral smectic C structure for the viral films. In chiral smectic C structures, the molecular long axis (director: n) have tilted angles (θ) with respect to layer normal (z). These tilted layers form a helical rotation (azimuth angle:Φ) from one layer to next layer, which is depicted in FIG. 16B.¹⁷ Therefore, the continuous helical change of the orientational orders through the tilted smectic layers cause different interaction with plane polarized light, and exhibit the optically active band patterns. Reflected polarized optical microscopy (RPOM) of the viral film give similar optically active dark and bright band patterns depending on the angles between the polarizer and analyzer. These RPOM images indicate the presence of dechiralization line defects^{17,35} on the surface. The dechiralization line defects arising from the interaction between helically ordered bulk structures and surface effects. Due to the surface effect, the helicoidal ordered chiral smectic C structures are unwound near the surface and result in bright and dark band patterns which correspond to the periodic pitch of chiral smectic C structures.

The dechiralization line defects of the viral film were characterized using scanning electron microscope (SEM) (FIG. 17B). Zig-zag patterned long-range ordered structures were observed, which corresponded to the dark and bright band patterns in RPOM. The alternating zig-zag band patterns (˜37 μm) showed periodic +45 degrees and −45 degrees changes with respect to the layer normal. The periodic spacing of the zig-zag patterns was consistent with the periodic POM and RPOM patterns. The zig-zag type morphologies of the viral film might be induced from surface defects of chiral smectic C structure of the viral film. The chiral smectic C structure has two ordering parameters, a tilted angle (θ) with respect to the layer normal and an azimuth angle (Φ) with respect to a layered plane.¹⁷ If the helicoidal pitch direction of the chiral smectic C layer is parallel with respect to the layer plane, the azimuth angle (Φ) of the director can be projected to the layered plane.¹⁸ Due to additional higher ordering properties on the surface, the tilted angle (θ) on the surface might have higher angles than the sum of the tilted angles and the projected azimuth angle.¹⁹ Therefore, the 180 degrees phase difference of the azimuth angle (Φ) is projected into the long-range periodic zig-zag patterns like FIGS. 16C and 17B.

Tilted smectic C morphologies on the free surface of the viral films were characterized using AFM (FIG. 17C). The M13 virus particles made tilt layer structure that had an average spacing of 620±27 nm. The molecular long-axis of the virus particles were tilted ˜45 degrees with respect to the layer normal (z). The distance measured through the director (n) between the adjacent two layers (886±36 nm) corresponded with the length scale of M13 phage particles (880 nm).²⁰ Based on the average layer spacing from the AFM image and chiral smectic C pitch from the POM image, the number of layers in a chiral smectic C pitch can be estimated to 59.3 layers. Because the azimuth angle changes 360 degrees in a pitch, it can be estimated from the number of the layers in a pitch (59.3 layers). The azimuth angle (Φ) from the viral film sample 1 was ˜6 degrees.

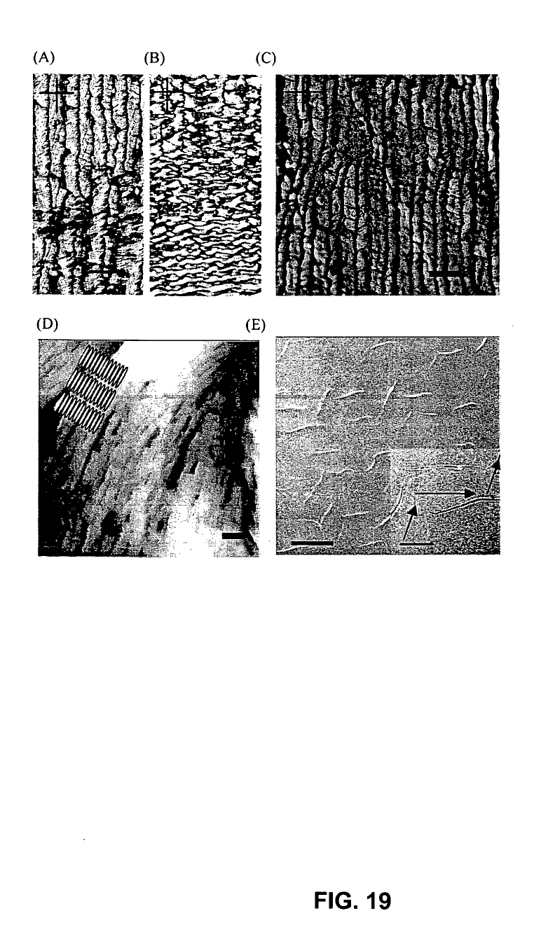

The helical periodic pitch of the viral film was also measured using laser light scattering. Clear diffraction patterns (FIG. 19E) gave a 35.8 μm pitch which is consistent with the periodic pattern from POM and SEM.

Distortion of the Chiral Smectic C Ordered Films:

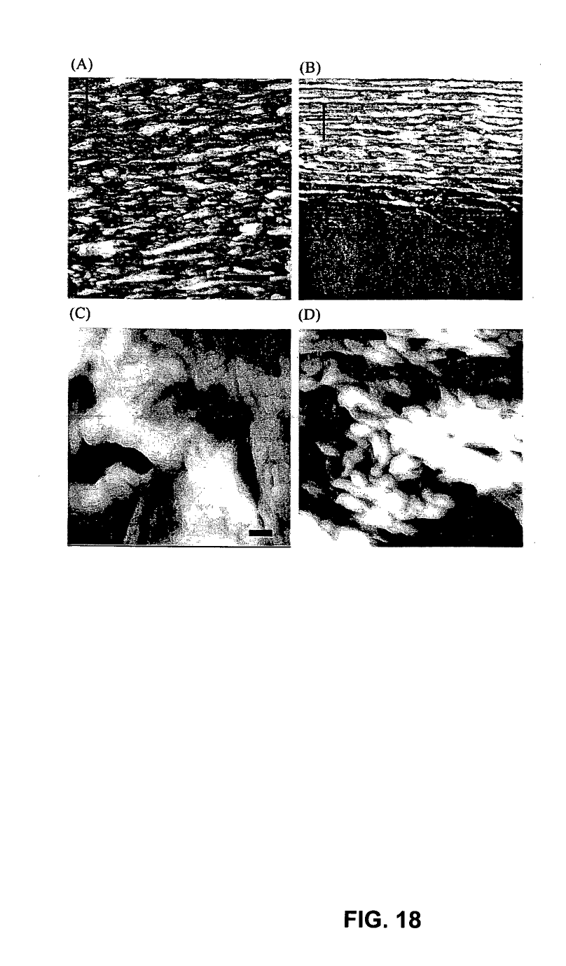

In certain regions of the film locally distorted textures were observed (FIG. 18A). In these disordered regions the band patterns were parallel to the ordered band patterns described previously. The spacing in these regions was observed to be irregular and varied. On the bottom part of the film (c area in FIG. 16A), grey band patterns emerged texture reported previously.²¹ Using AFM, twisted deformations of smectic A structures were observed on these distorted band texture areas. AFM images (FIG. 18C) showed that smectic layers were twisted and formed the disclination line which showed the discontinuity of the orientation. These chiral smectic A POM textures and twisted smectic layers morphologies suggested that chiral smectic C structure might transition to a twisted grain boundary (TGB) structure that is known to form between a chiral smectic C and an isotropic phase.²¹ AFM images collected from the grey POM region (FIG. 18B) showed irregular distorted smectic C domains. However, when a differential interference contrast (DIC) filter was applied to this grey pattern texture area, the periodic band patterns, which were similar to the chiral smectic C periodic patterns, were observed. These periodic DIC images and distorted AFM morphologies indicated that the grey pattern areas might have chiral smectic C structures in the bulk and the distortion might be localized on the surface areas.

The viral film characteristics, which were fabricated from concentration range 6.38-9.70 mg/ml (sample 2˜7), were similar to the viral film (sample 1) fabricated from 9.93 mg/ml described above. The pitch length gradually decreased from 9.93 to 7.60 mg/ml and increased until 5.09 mg/ml. At this concentration (5.09 mg/ml), the smectic C structure made a transition to smectic A structure. A similar expansion of the pitch near the transition point was also observed from the cholesteric phase transition to the smectic phase.²² Therefore, the chiral smectic C spacing expansion might be involved with a pre-transition phenomena. All of the films showed clear diffraction patterns which were consistent with periodic patterns in POM (table. 2).

Structure Transition:

Different POM band patterns (upper part of FIG. 4A) were observed from the viral film fabricated from a concentration of 5.09 mg/ml (sample 7). POM images of sample 7 exhibited periodic vertical bright band patterns which were divided by schlieren stripe lines when the dark lines were parallel with respect to the polarizers. When the analyzer angle changed by around five degrees, the POM texture intensity changed to slightly darker and brighter stripe patterns similar to the chiral smectic C viral film. The film also exhibited zig-zag patterned lines through the band patterns. The periodicity of these vertical periodic patterns was 97.43±2.92 μm. When the samples are rotated through the optical axis, bright band patterns were changed to alternating dark and bright stripe patterns. The intensity dependence on both the change of angles between polarizers and the rotation change of the orientation on the film surface.

Gradual changes of the POM textures (bottom part of FIG. 4A and upper part of FIG. 19B) were observed on the middle part (b area in FIG. 16A) of the sample surface (5.09 mg/ml). The vertical stripes patterns gradually transitioned to parallel dark and bright stripe patterns (bottom part of FIG. 19B) in sample 1-6. The parallel stripe patterns had 41.04±2.18 μm periodicity. Unwinding defects of the chiral smectic C structure were observed where the vertical stripes met the parallel stripes. Schlieren line texture was propagated parallel to the direction of meniscus force. Sample areas near the bottom part of the film exhibited the grey textures which were observed in sample 1.

Smectic A Ordered Films:

POM images of sample 8-10 (4.39-2.59 mg/ml) exhibited the same vertical bright band patterns (FIG. 19C) observed in the sample 7. However, spacing between the two vertical dark lines was varied as showed in table 2. The long-range periodic zig-zag patterns on the surface were also characterized using SEM.

The low magnification SEM image (FIG. 19C) from sample 10 showed that the film had regularly occurring periodic chevron-like cracked patterns. The higher magnification SEM image (inset of FIG. 19C) of these cracked pattern showed that their directions were parallel with respect to the orientation of the directors. Between the interfaces of zig-zag patterns, the disclination lines were observed to correspond to the dark vertical schlieren line patterns in the POM images (FIG. 19C). Using AFM, smectic A ordered structures were observed in the same region (FIG. 19D). The viral particles formed ˜980×800 nm domain blocks. In the smectic domains, the virus particle packing pattern was close to the smectic B structure in which molecules are arranged in layers with the molecular center positioned in a hexagonal close-packed array. These domain blocks formed the parallel-aligned and bookshelf-like smectic A structures on the surface. The average spacing between the two layers measured was 977±25 nm which is slightly larger than the length of M13 virus.

Nematic Ordered Films:

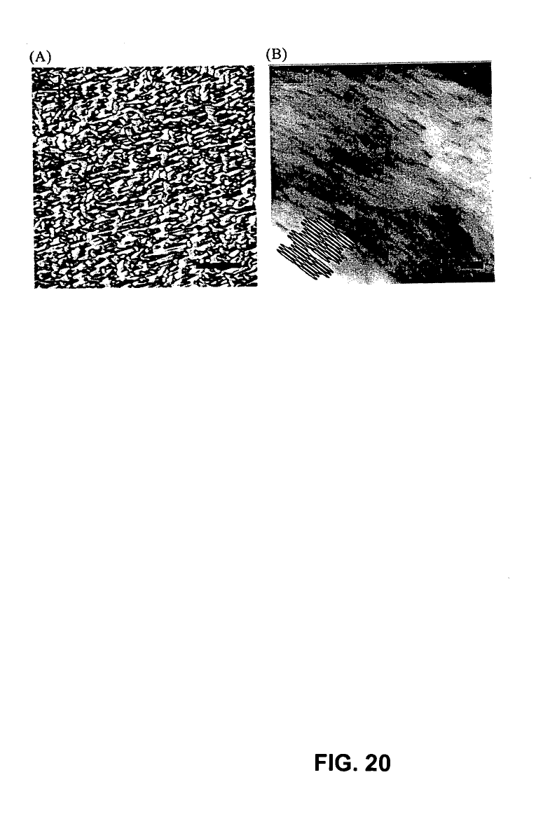

POM image of sample 11 showed the disordered schlieren texture lines (FIG. 20A). Crooked black brush line patterns propagated irregularly within 20˜30 micrometer domains. The dark and bright patterns were divided by the crooked black brush lines. Both the dark brush lines and the brightness of the patterns were changed by rotating the film indicating that these brush lines were disclination lines. AFM images of these areas showed the nematic ordered structures of smectic A bundle-like domains (˜980 nm×200 nm)(FIG. 20B). Each smectic A domain formed nematic like ordered structures which oriented through the molecular long axis as the preferred direction.

Chirality Consideration

Meyer first suggested the chiral smectic C structure.¹⁴ He predicted if smectic C structures were formed from chiral molecules, the resulting structure should be a chiral smectic C structure. Many chiral thermotropic liquid crystalline materials have been synthesized that have the chiral smectic C structures.^{17,23,24} However, due to the non-uniform orientation of the lyotropic liquid crystals, it has been challenging to study chirality effects of lyotropic smectic structures compared with those of thermotropic liquid crystals. Chirality of the lyotropic smectic liquid crystals has been reported.^{20,25,26} A twisted grain boundary phase of the Fd virus was observed.²⁰ Although optical microscopy evidence of the chiral smectic phase (SmC*, SmI*, SmF*) of filamentous actin (F-actin) was reported, long-range ordering of chiral smectic C structure of F-actin could not be observed due to the polydisperse nature of F-actin.²⁵ Moreover, making a long-range ordered lyotropic liquid crystalline structure without an external field has proven difficult. Long-range ordered samples, such as viral fibers and suspensions, can be prepared from the external field effect.^{27,28} However, these samples lose their chiral properties in response to the external fields. Viral films fabricated from the monodisperse M13 phage studied in this paper exploited the meniscus force in order to make the long-range ordered chiral smectic C structure up to several centimeters in length without external fields. POM images of the viral film showed optically active dark and bright stripe patterns. SEM images showed the dechiralization defects of chiral smectic C structures. AFM images showed the tilted smectic C ordered structures. Based on these microscopic evidences, it was concluded that the viral films have the chiral smectic C structure.

Thickness effects of the chiral smectic C structure of the viral film was also observed. When the thickness of the film decreases to ˜4.3 μm, which had ˜360 viral particle layers (particle to particle distance: 12 nm)⁵, the surface effect seemed to be dominant throughout the bulk film. Therefore, the chiral smectic C structure made a transition to a smectic A like ordered structure. The orientation of the molecular long-axis was almost perpendicular with respect to the smectic layers. However, the zig-zag like periodic patterns were still observed. The formation of the vertical zig-zag patterns as observed from sample 7 to sample 10 might come from both the helical structure of the bulk and thickness of the film. Due to the thickness effects, relatively thin viral films (2˜4 micrometer in thickness) aligned in smectic A patterns, which is similar to the thin nematic films that have smectic like ordered structures¹⁹. The intrinsic chiral properties of the virus which forms layers might stabilize the zig-zag patterned smectic A structure instead of a bookshelf like smectic A patterned structure.

The mechanism for the self-ordered virus film formation is still under investigation. The nematic ordered structures, which showed the disordered smectic A domains, strongly suggested the formation of bundle-like domains in solution prior to the film formation. The isotropic phase of the viral suspension in the meniscus areas slowly made a transition to the nematic phase. However, viral particles that have the same orientational order began to make bundle-like domain structures. These domain structures are still flexible to modification of their packing structure. These domains initially become the basic building units of the layered structures. After forming layers, these smectic A domains become close-packed as the solvent evaporates. Complete evaporation of the solvent forms the bulk structures of the viral film. The thickness of the virus film has a critical effect on both the bulk and surface structure. Surface forces are dominated in the formation of the thin virus films. These interactions force the bundle-like domains to be aligned in smectic A patterns. However, in the thick viral films (more than 360 layers of the viral layer) surface morphologies are affected by both surface forces and the bulk chiral structure. Therefore, the smectic C patterns are dominant compared with the smectic A morphologies in the thin samples. Bundle-formation phenomena in experiments involving cast films of liquid crystals have also been observed.²⁹⁻³¹ From M13 viral films formed on mica, SiO₂, and silicon substrates, the M13 bundles were frequently observed at the initiation of film formation and thought to act as nucleation centers for oriented deposition of viruses on these substrates²⁹.

The morphologies of the ZnS nanocrystal virus hybrid films were previously reported⁵. The ZnS nanocrystal hybrid viral films have the optically active ˜72 μm periodic dark and bright stripe POM patterns which were similar to that of 100% M13 virus films. However, the surface morphologies of the ZnS nanocrystal hybrid viral films have anti-smectic C structures (smectic O), which appear in a zig-zag pattern that have ˜1.0 μm spacing through the layer normal direction. Based on the POM pitch and AFM zig-zag layer spacing, the ZnS nanocrystal hybrid viral films have ˜72 layers in a pitch and ˜5 degrees in azimuth angle. Based on these 100% M13 virus control films and the surface morphologies found from the ZnS nanocrystal hybrid viral films, it can be concluded that the ZnS nanocrystal hybrid viral films have chiral smectic C structures which are composed of interdigitated domains of M13 viruses bound to 20 nm ZnS nanocrystal aggregates. The interdigitated domains can reduce the packing energies of the big head shape of the ZnS nanocrystal hybrid viral films. The anti-smectic C structure was generally only observed on the surface of the film and generally believed to be a surface effect.

The observed morphologies of the M13 viral films and ZnS nanocrystal hybrid viral films were very similar with those of rod-like polymer (poly (γ-benzyl α, L-glutamate), (PBLG)) and rod-coil block-copolymers, which is approximately a thousand times smaller than the virus system.^{4,32,33} Monodisperse rod-like polymers have been known to form smectic film structures.³² The high ratio rod-coil (f_{rod-coil}>0.96) block-copolymers favor the bilayered and interdigitated morphologies, which exhibit smectic C and O structures.⁴ A TGB structures of a PBLG film made of monodisperse PBLG was reported to have a chiral smectic structure.³³ The same film formed using technique of this invention may yield a chiral smectic C structure and therefore also support Meyer's prediction.

Using external force such as a magnetic field or an electric field can aid, for example, in building defect free and well ordered miniaturized electronic devices using these genetically engineered virus based films after hybridization of the viruses with semiconductor or magnetic nanocrystals. Homeotropic-aligned magnetic nanocrystals hybrid virus thin films can be used, for example, for self-supporting, flexible, and highly integrated magnetic memory devices.

References for Additional Description and Working Examples (Embodiment A):

1. Huang, Y.; Duan, X.; Lieber, C. M. Science 2001, 291, 630-633.

2. Fowler, C. E.; Shenton, W.; Stubbs, G.; Mann, S. Adv. Mater. 2001, 13, 1266-1269.

3. Mathias, J. P.; Simanek, E. E.; Whitesides, G. M. J Am. Chem. Soc. 1994,116, 4326-4340.

4. Chen, J. T.; Thomas, E. L.; Ober, C. K.; Mao, G.-P. Science 1996, 273, 343-346.

5. Lee, S.-W.; Mao, C.; Flynn, C. E.; Belcher, A. M. Science 2002, 296, 892-895.

6. Boal, A. K.; Ilhan, F; DeRouchey, J. E.; Thum-Albrecht, T.; Russell, T. P.; Rotello, V. M. Nature 2000, 404, 746-748.

7. Mirkin, C. A.; Letsinger, R. L.; Mucic, R. C.; Storhoff, J. J. Nature 1996, 382,607-609.

8. Mao, C.; Flynn, C. E.; Hayhurst, A.; Sweeney, R.; Qi, J.; Williams, J.; Georgiou, G.; Iverson, B.; Belcher, A. M., Nature submitted.

9. Douglas, T.; Young. M. Nature 1997, 393, 167-170.

10. Adams, M.; Dogic, Z.; Keller, S. L.; Fraden, S. Nature 1998, 393, 349-352.

11. Hartgerink, J. D.; Beniash, E.; Stupp, S. I. Science 2001, 294, 1684-1688.

12. Winfree, E.; Liu, F.; Wenzler, L. A.; Seeman, N. C. Nature 1998, 394, 539-544.

13. Cha, J. N.; Shimizu, K.; Zhou, Y.; Christiansen, S. C.; Chmelka, B. F.; Stucky,G. D.; Morse, D. E. Proc. Nation. Acad. Soc. 1999, 96, 361-365.

14. Meyer, R. B.; Leibert, L.; Strzelecki, L.;.Keller, P. J. Phys. (Paris) Lett. 1975, 36, 1-69.

15. Patrick, D. L.; Cee, V. J.; Morse, M. D.; Beebe, T. P. J. of Phys. Chem. B 1999, 103, 8328-8336.

16. Walba, D. M.; Stevens, F.; Clark, N. A.; Parks, D. C. Acc. Chem. Res. 1996, 29, 591-597.

17. Clark, N. A. Ferroelectric Liquid Crystals: Principles, Properties, and Applications; Gordon and Breach Science Pub.; Philadelphia, Pa. 1991.;

18. Clark. N. A., Rieker, T. P, Phys. Rev. A, 1988, 37,1053-1056.

19. Sonin, A. A.; Clark, N. A., Freely Suspended Liquid Crystalline Films; John Wiley & Sons, Ltd, New York, 1998, pp. 25-43 and pp. 75-78.

20. Baus, M.; Rull, L. F.; Ryckaert, J. Observation, prediction and simulation of phase transitions in complex fluids; Kluwer Academic Pub.; Boston:, 1995 (113-164).

21. Goodby, J. W.; Waugh, M. A.; Stein, S. M.; Chin, E.; Pindak, R.; Patel, J. S. J. Am. Chem. Soc., 1989, 111, 8119-8125.

22. Dogic, Z.; Fraden, S. Langmuir 2000, 16, 7820.

23. Fukuda, A.; Takanishi, Y.; Isozaki, T.; Ishikawa, K.; Takezoe, H. J. Mater. Chem. 1994, 4, 997-1016.

24. Zheng, W. Y.; Albalak, R. J.; Hammond, P. T. Macromolecules, 1998, 31, 2686-2689.

25. Das, P.; Xu, J, Roy. 3; Chakrabarti, N. J. Chem. Phys. 1999, 111, 8240-8250.

26. Kamien, Randall D.; Lubensky, T. C. J. de Physique II 1997, 7, 157-163.

27. Welsh, L. C.; Symmons, M. F.; Nave, C.; Perham, R. N.; Marseglia, E. A.; Marvin D. A. Macromolecules, 1996, 29, 7025-7083.

28. Booy, F. P.; Fowler, A. G. Int. J. Biol. Macromol. 1985, 7, 327.

29. Ni, J.; Lee, S.-W.; White, M. J.; Belcher, A. M. (unpublished data).

30. Maeda, H.; Maeda, Y. Langmuir 1996, 12, 1446-1452.

31. Maeda, Y.; Hachisu, S. Collids Surf. 1983, 6, 1

32. Yu, S. M.; Conticello, V. P.; Zhang, G.; Kayser, C.; Fournier, M. J.; Mason. T. L.; Tirrell, D. A. Nature 1997, 389, 167-170.

33. He, S.-J.; Lee, C.; Gido, S. P.; Yu. S. M.; Tirrell, D. A. Macromolecules 1998, 31, 9387-9389.

34. Wetter, C., Biologie in unserer Zeit 1985, 3, 81-89.

35. Glogarova, M.; Lejek, L; Pavel, J; Janovec, U; Fousek, F; Mol. Cryst. Liq. Cryst. 1983, 91, 309-325.

### ADDITIONAL DESCRIPTION AND WORKING EXAMPLES (EMBODIMENT B)

The paper by Lee et al. “Virus-Based Alignment of Inorganic, Organic, and Biological Nanosized Materials” Advanced Materials, 2003, 15, 9, 689-692 is incorporated by reference in its entirety including figures, experimental, and results and discussion.

Additional materials were prepared which can be used as films in storage applications, as well as other applications. In an additional embodiment, a new platform is presented for organization of a variety of materials including inorganic nanoparticles, small organic molecules and large biomolecules that organize and self-assemble at the nanometer length scale and are continuous into the centimeter length scale. Long-range ordered nano-sized materials (10 nm gold nanoparticles, fluorescein, phycoerythrin protein) were fabricated using a streptavidin linker and anti-streptavidin M13 bacteriophage (virus). The anti-streptavidin viruses, which formed the basis of the self-ordering system, were selected to have a specific recognition moiety for streptavidin using phage display. The nano-sized materials were previously bound to streptavidin. Through the molecular recognition of the genetically selected virus, the nano-size materials were bound ands n evolved into a self-supporting hybrid film.

Functionalized liquid crystalline materials can provide various pathways to build well-ordered and well-controlled two and three-dimensional structures for the construction of next generation optical, electronic and magnetic materials and devices.^{[1-3]} It has been demonstrated that several types of rod-shape viruses form well controlled liquid crystalline phases.^{[4,5]} Recently, a self-assembled ordered nanocrystal film fabrication method was reported using nanocrystal-functionalized M13 virus. ^{[3]} Through the utilization of genetic engineering techniques, one-end of the M13 virus was functionalized to nucleate or bind to a desired semiconductor material. These nanocrystal-functionalized viral liquid crystalline building blocks were grown into ordered hybrid self-supporting films. The resulting nanocrystal hybrid film was ordered at the nanoscale and at the micrometer scale into 72 μm periodic striped pattern domains. In the previous system, one could easily nucleate and align the nanoparticles for the II-VI semiconductor materials in an one-pot synthetic route. In order to align other materials including metals and electro-optical materials, biological selection and further evolution are required for each material prior to aligning the reported using anti-streptavidin viruses, where the virus was first selected to bind streptavidin protein units. This allowed for a universal handle for the virus to pick up any material that has been covalently conjugated to streptavidin. Then the self assembling nature of this anti-streptavidin virus can be exploited to make organized hybrid materials. The organized hybrid materials presented here are liquid crystalline films of gold nanoparticles, fluorescent molecules (fluorescein) and large fluorescent proteins (phycoerythrin).

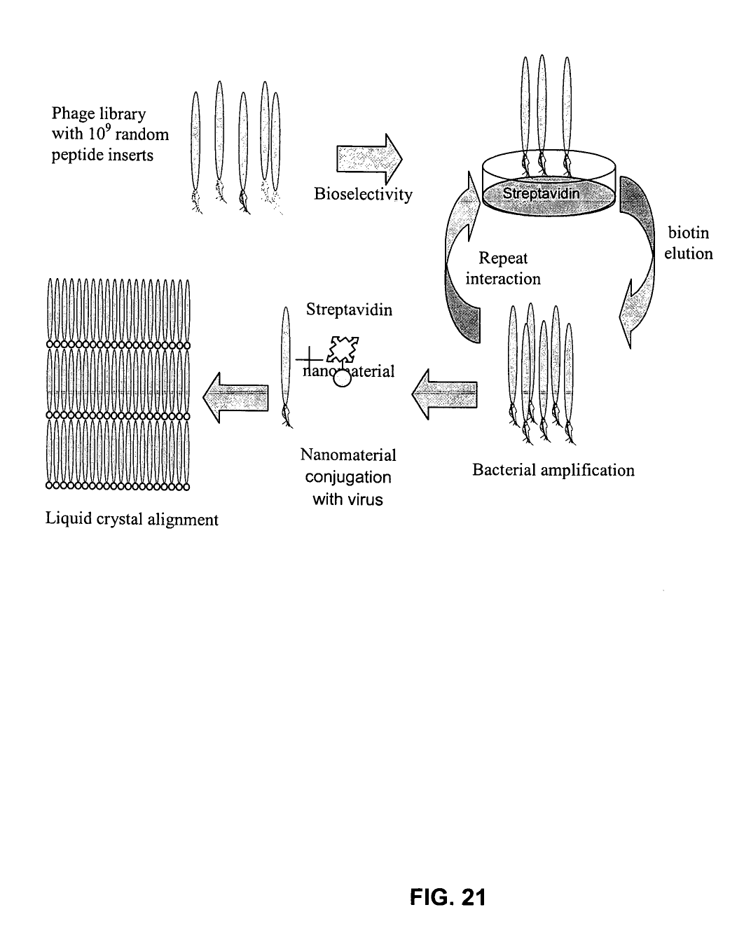

The anti-streptavidin M13 viruses having specific binding moieties for the streptavidin were isolated through the screening of a phage display library (FIG. 21).^{[6,7}] Streptavidin has the known specific binding motif His-Pro-Gln.^{[6]} His-Pro-Gln sequences were isolated as pIII inserts after second round selection of phage for the streptavidin target. His-Pro-Gln binding motif made up 100% of the pIII insert after fourth round selection and sequencing. The dominant binding sequence after the fourth round was TRP ASP PRO TYR SER HIS LEU LEU GLN HIS PRO GLN. This anti-streptavidin M13 virus was amplified to high concentration (˜10¹² pfu) and reacted with 10 nm gold nanocrystals (FIG. 2A), fluorescein, and phycoerythrin which were previously conjugated with streptavidin. These highly concentrated suspensions exhibited liquid crystalline properties.

The highly concentrated Au-virus liquid crystalline suspension (˜83 mg/ml) exhibited an iridescent birefringence texture when analyzed using polarized optical microscopy (POM) (FIG. 2B). This iridescent birefringence texture corresponded to a smectic liquid crystalline phase structure. The cholesteric finger print textures (76˜20 mg/ml) and nematic textures (14 mg/ml) were observed when the suspension were systematically diluted.

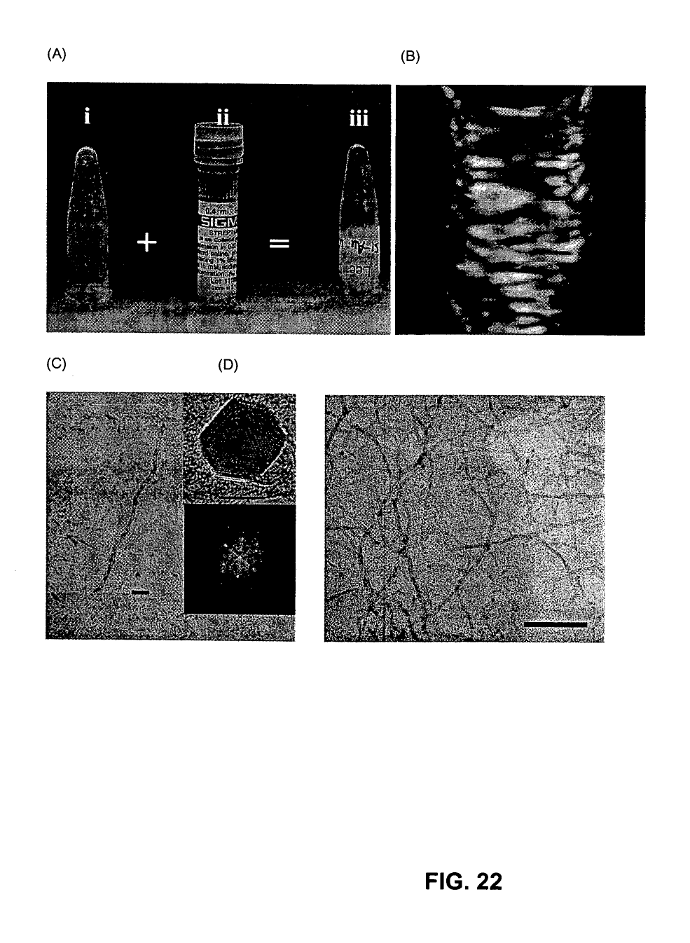

The individual mesogen units of 10 nm gold nanoparticles bound viruses were visualized using transmission electron microscopy (TEM) prior to staining with 2% uranyl acetate. These individual Au and virus complex (Au-virus) were isolated from 0.01% dilution of the smectic phase suspension (FIG. 22C). In the 0.1% dilution, aggregation of Au-virus complex were observed (FIG. 22D). Most mesogen units observed had one virus bound to one 10 nm Au particle at the pIII end of virus. However, both unbound gold nanoparticles and unbound viruses were observed in less than 20% of mesogen units. In addition, two gold nanoparticles bound with one virus and one gold nanoparticle bound with two viruses were also observed (less than ˜5%). These undesired binding behaviors between viruses and streptavidin conjugated gold nanoparticles may be caused by a mismatch in numbers of the recognition groups between the viruses and streptavidin. The M13 virus has five pIII streptavidin-recognition units at the end of virus and the streptavidin is known to have four binding sites for the biotin. ^{[8]} Due to empirical stoichiometric control and steric effects, mesogen units could be constructed where the majority of the population contained one virus with one Au nanoparticle.

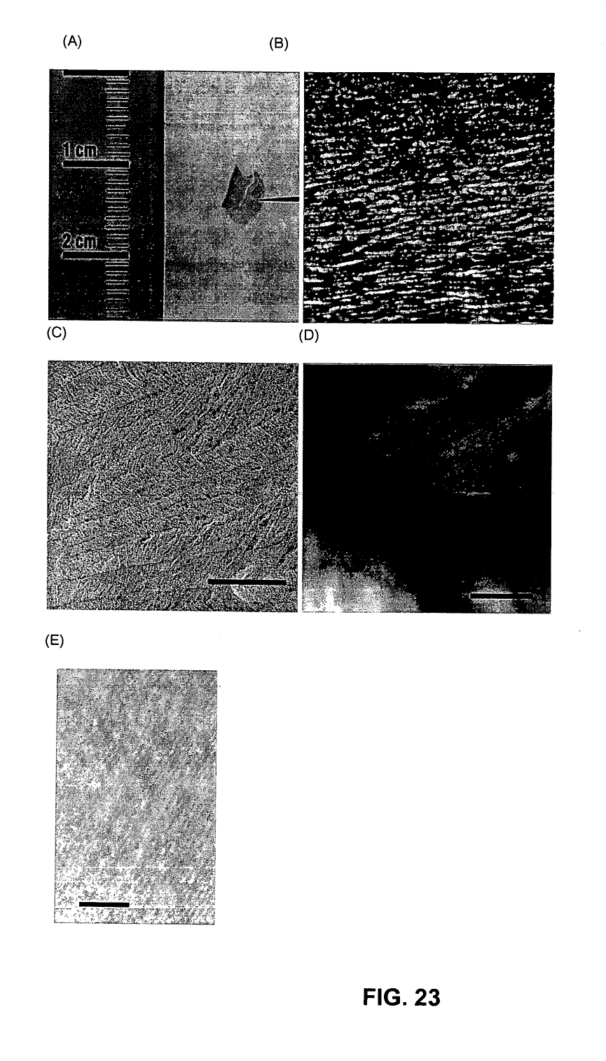

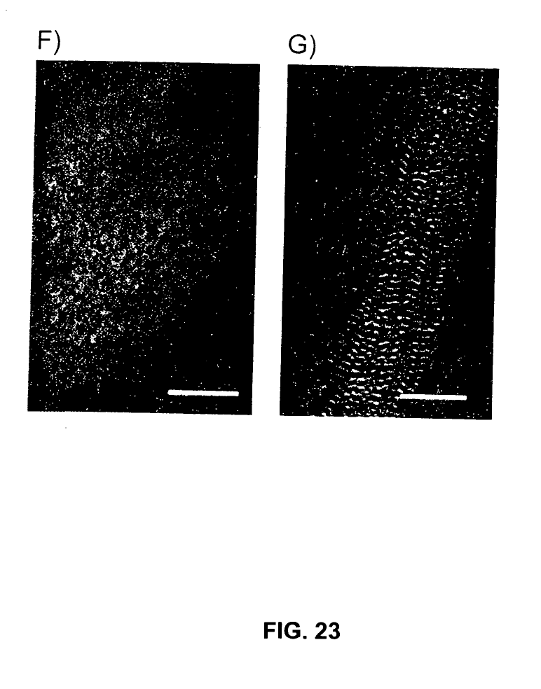

Smectic ordered self-supporting Au-virus films (FIG. 23A) were prepared from a dilute Au-virus solution (˜6 mg/ml). The viruses and nanocrystals were agitated for one week prior to the fabrication of the film. The suspension was kept dry in a dessicator for two weeks. The viral nanocrystal hybrid film was slightly pink in color and transparent. The ordered morphologies of the viral film were characterized by POM, scanning electron microscopy (SEM) and atomic force microscopy (AFM). The thickness of the film was 5.68±0.65 μm.

Optical characterization revealed that the films were composed of ˜10-μm dark-grey periodic horizontal striped patterns (FIG. 23B). These stripes were optically active and changed their bright and dark patterns depending on the angles between a polarizer and an analyzer. These striped patterned POM characteristics are similar to the smectic virus films that were previously reported by our group. ^{[9]} Surface morphologies of these striped patterns were characterized using SEM. SEM images (FIG. 23C) showed that the Au-virus hybrid film had long range ordered zig-zag periodic morphologies that were composed of ten to twelve smectic layers in a periodic pattern. The average spacing of zig-zag periodic bands, which corresponded to one chiral smectic C pitch of the typical virus film ^{[9]}, was 9.34±0.78 μm. AFM images (FIG. 23D) showed that the hybrid film has a smectic C structure. The average layer spacing between two adjacent layers was 833±12 nm. Layer spacing measured through the molecular long axis was 977±65 nm. The average tilted angle was ˜54 degrees with respect to the layer normal. The length of the M13 virus is 880 nm. This ˜100 nm longer spacing observed through the molecular long axis is strong evidence to support an interdigitated structure. ^{[10]} The shape of mesogen unit which has a big head (inorganic gold nanoparticle) with a long tail (organic M13 virus) might have lower packing free energy by forming interdigitated structures. Additionally, the ˜10 μm periodic zig-zag patterns observed in POM and SEM images highly indicated that the Au-virus hybrid films also have chiral smectic C structure in the bulk and dechiralization defects on the surface of the hybrid films.

Two kinds of organic materials were also fabricated in virus films. The organic materials were chosen to show that this technique is versatile but these materials also allow easy visualization of the approximately one micrometer periodic long ranged ordering because they are fluorescent. Thin cast films of virus bound fluorescein and phycoerythrin were fabricated using streptavidin and anti-streptavidin M13 viruses. Due to the enhanced ordered properties of liquid crystalline materials near the surface and capillary driving force during the drying process, the smectic layer structure was easily observed from drop-cast thin films of fluorescein complex viruses (F-virus) and phycoerythrin complex viruses (P-virus) (FIG. 23E). The ordering of these liquid crystalline hybrid materials were enhanced by casting thin films of these materials. In similar phenomena, nematic liquid crystalline materials formed surface stabilized smectic phase due to the surface effects ^{[11]} and chiral smectic C structures transitioned into smectic A structures ^{[9]} in thin films. Scanning laser microscopy was used to optically section the F-virus thin films (FIG. 23F). These thin films showed weak stripe patterns which corresponded to a smectic structure. Applying similar analysis to the thin film of fluorescent P-viruses (FIG. 23G) very clear one micrometer stripe patterns were observed. These one micrometer fluorescence patterns indicated that the fluorescent molecules (fluorescein and phycoerythrin) were bound to the viruses by the linkage of streptavidin, then formed the smectic layer structures. Because the fluorescent materials were imposed at the end of the virus, their position was localized between the smectic layer interface boundaries.

In this invention, anti-streptavidin M13 viruses were used to self-assemble various nano-sized materials. The anti-streptavidin M13 viruses provide a convenient method to organize a variety of nano-sized materials into self-assembled ordered structures. Because the modification of the DNA insert allows for controlled modification of the virus length, the spacing in the smectic layer can be genetically controlled. ^{[12]} By conjugating other nano-sized materials (magnetic nanoparticles, II-VI semiconductor nanoparticles, functional chemicals, etc) with streptavidin, this anti-streptavidin method can align various nano-sized materials at the desired length scale which is defined by the smectic layers.

Experimental:

The anti-streptavidin virus was selected by a phage display method using a M13 bacteriophage library (New England Biolab). The virus was amplified in a large volume (400 ml scale, 7×10⁷pfu). The virus suspension was precipitated into a pellet. 20 mg of the virus pellet was suspended with 1.0 ml of 10 nm gold nanoparticle (Abs: 2.5 at 520 nm), conjugated with a streptavidin colloidal suspension (Sigma Co.), and agitated using a rocker for one day. The viruses conjugated with gold nanoparticles (Au-virus) were centrifuged after adding 167 μl of poly ethylene glycol solution. The red colored pellet was suspended using ˜20 μl of tris-buffered saline solution (pH 7.5) to form Au-virus liquid crystalline suspension (virus concentration: 83.2 mg/ml). In order to fabricate the Au-virus film, the Au-virus suspension was diluted to ˜6 mg/ml (400 μl) and kept dry in a dessicator for two weeks.

Fluorescein-virus Cast Film Fabrication:

20 μl of virus suspension (1.9×10⁻⁷M in Tris-HCl saline buffered solution (pH 7.5)) was mixed with 20 μl of 0.01 mg/ml (1.9×10⁻⁷ M, MW: 53,200) of fluorescent-streptavidin suspension. 1 μl of suspension was cast and dried on the glass substrate. The molarity of virus suspension was measured using UV-Vis spectrophotometer (extinction coefficient: 1.2×10⁸ M⁻¹cm⁻¹ at 268 nm). ^{[13 ]}

Phycoerythrin-virus and Cast Film Fabrication:

20 μl of the virus suspension (˜6 mg/ml, 1.9×10⁻⁷M, MW: 292,800 Tris-HCl saline buffered solution (pH 7.5)) was mixed with 20 μl of 0.05 mg/ml (1.7×10⁻⁷ M in Tris-HCl saline buffered solution (pH 7.5) with 5% sucrose) of R-phycoerythrin-streptavidin. 1 μl of suspension was cast and dried on the glass substrate.

Microscopy:

POM images were obtained using Olympus polarized optical microscope. Micrographs were taken using SPOT Digital camera (Diagnostic Inc.). Scanning laser microscopy images was obtained using Leica TCS 4D and SEM images were obtained using LEO1530, operating at an accelerating voltage of 1 KV. TEM images were obtained using Philips 208 at an accelerating voltage of 80 kV and a JEOL 2010F at 200 kV. The AFM images (Digital Instruments) were taken in air using tapping mode. The AFM probes were etched silicon with 125 μm cantilevers and spring constants of 20-100 N/m driven near their resonant frequency of 250-350 kHz. Scan rates were of the order of 1-40 μm/s.

References for Additional Description and Working Examples (Embodiment B):

1. L. Li, J. Walda, L. Manna, A. P. Alivisatos, Nano Letters 2002, 2, 557.

2. V. Percec, M. Glodde, T. K. Bera, Y. Miura, I. Shiyanovskaya, K. D. Singer, V. S. K. Balagurusamy, P. A. Heiney, I. Schnell, A. Rapp, H.-W. Spiess, S. D. Hudson, H. Duan, Nature 2002, 419, 384

3. S.-W. Lee, C. Mao, C. E. Flynn, A. M. Belcher, Science 2002, 296, 892.

4. Z. Dogic, S. Fraden, Phys. Rev. Lett. 1997, 78, 2417.

5. Z. Dogic, S. Fraden, Langmuir 2000, 16, 7820 (2000).

6. J. J. Devlin, L. C. Panganiban, P. E. Devlin, Science 1990, 249, 404.

7. S. R. Whaley, D. S. English, E. L. Hu, P. F. Barbara, A. M. Belcher, Nature 2000, 405, 665.

8. P. C. Weber, D. H. Ohlendorf, J. J. Wendoloski, F. R. Salemme, Science 1989, 243, 85.

9. S.-W. Lee, B. M. Wood, A. M. Belcher, Langmuir 2002, 19, 5, 1598.

10. J. T. Chen, E. L. Thomas, C. K. Ober, G.-P. Mao, Science 1996, 273, 343.

11. A. A. Sonin, N. Clark, Freely Suspended Liquid Crystalline Films, John Wiley & Sons, Ltd, New York, 1998, pp. 25-43.

12. Z. Dogic, S. Fraden, Phil. Trans. Roy. Soc. London:A 2001, 359, 997.

13. T. A. Roth, G. A. Weiss, C. Eigenbrot, S. S. Sidhu, J. Mol. Biol. 2002, 322, 357.

Although this invention has been described in reference to illustrative embodiments, this description is not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

Citation to references herein does not constitute any admission that these references are prior art to the present invention.

What is claimed is:

1. A fabricated biofilm storage device for long term storage of biological material comprising:
optionally, a substrate having a contacting surface, and
a biologic material on the optional contacting surface and forming a stable film, wherein the film is stable at room temperature for at least 7 weeks.

2. The fabricated biofilm storage device of claim 1, wherein the stable film is stable for at least five months based on time dependent infection ability in the film state.

3. The fabricated biofilm storage device of claim 1, wherein the stable film is stable at room temperature for at least six months.

4. The fabricated biofilm storage device of claim 1, wherein the substrate is present and chosen from the group consisting of Langmuir-Blodgett films, functionalized glass, germanium, silicon, a semiconductor material, PTFE, polycarbonate, mica, mylar, protein film, plastic, quartz, polystyrene, gallium arsenide, gold, silver, metal, metal alloy, fabric, mammalian tissue, and combinations thereof.

5. The fabricated biofilm storage device of claim 1, wherein the stable film is dry.

6. The fabricated biofilm storage device of claim 1, wherein the stable film is self-supporting.

7. The fabricated biofilm storage device of claim 1, wherein the stable film comprises, in addition to the biological material, one or more organic or inorganic molecules.

8. The fabricated biofilm storage device of claim 7, wherein an organic molecule is present and is chosen from the group consisting of carbon, single stranded nucleic acid, double stranded nucleic acid, peptide, protein, antibody, enzyme, steroid, drug, chromophore, conducting polymer, vaccine, and combinations, thereof.

9. The fabricated biofilm storage device of claim 7, wherein an organic molecule is present and is chosen from the group consisting of protein, enzyme, drug, and combinations thereof.

10. The fabricated biofilm storage device of claim 7, wherein an inorganic molecule is present and is chosen from the group consisting of indium tin oxide, a doping agent, metal, metal alloy, mineral, semiconductor, and combinations thereof.

11. The fabricated biofilm storage device of claim 1, wherein the biologic material is chosen from the group consisting of a virus, bacteriophage, bacteria, peptide, protein, antibody, enzyme, amino acid, steroid, drug, carbohydrate, lipid, chromophore, single-stranded or double-stranded nucleic acid, vaccine, and chemical modifications thereof.

12. The fabricated biofilm storage device of claim 1, wherein the biological material is a virus or bacteriophage.

13. The fabricated biofilm storage device of claim 1, wherein the biological material is a bacteria.

14. The fabricated biofilm storage device of claim 1, wherein the biological material is a peptide or protein.

15. The fabricated biofilm storage device of claim 1, wherein the biological material is an antibody or enzyme.

16. The fabricated biofilm storage device of claim 1, wherein the biologic material self-assembles to form a uniform thin film.

17. The fabricated biofilm storage device of claim 1, wherein the biological material is anisotropic.

18. The fabricated biofilm storage device of claim 1, wherein the biological material further comprises a vaccine.

19. The fabricated biofilm storage device of claim 1, wherein at least two biological materials are present.

20. The fabricated biofilm storage device of claim 1, wherein the biological material further comprises an inorganic nanoparticle.

21. The fabricated biofilm storage device of claim 7, wherein the one or more organic or inorganic molecules are preincubated with the biologic material.

22. The fabricated biofilm storage device of claim 21, wherein preincubation permits the formation of nanocrystals.

23. The fabricated biofilm storage device of claim 1, wherein the film exhibits biologic, optical, electrical, magnetic properties, or combinations thereof.

24. The fabricated biofilm storage device of claim 1, wherein the stable film is used in diagnosis, screening, analysis, testing, information gathering, data processing, drug discovery, microelectronics, optics, data storage, research, or combinations thereof.

25. The fabricated biofilm storage device of claim 1, wherein the structure of the stable film is controlled by solvent concentration, magnetic field, electric field, optics, and combinations, thereof.

26. The fabricated biofilm storage device of claim 1, wherein the biologic material is genetically engineered.

27. The fabricated biofilm storage device of claim 1, wherein the biofilm is stabilized with the addition of a storage solution.

28. The fabricated biofilm storage device of claim 1, wherein the biofilm is stabilized with the addition of a sugar-containing storage solution.

29. The fabricated biofilm storage device of claim 1, wherein the stability is monitored with use of light properties.

30. A method of fabricating a biofilm storage device comprising the steps of:
applying a biologic material to a substrate with a contacting surface, wherein optionally the contacting surface promotes uniform alignment of the biologic material on the contacting surface; and
allowing the formation of a stable film which is stable at room temperature for at least seven weeks.

31. The method of claim 30, wherein the stable film is dry.

32. The method of claim 30, wherein the biological material is a combinatorial library.

33. The method of claim 30, wherein the biologic material self assembles to form a thin film about 25 microns or less.

34. The method of claim 30, wherein uniform alignment is controlled by solvent concentration, magnetic field, electric field, optics, or combinations, thereof.

35. The method of claim 30, wherein fabricating the biofilm storage device is reversible.

36. The method of claim 30, wherein the biologic material is chosen from the group consisting of a virus, bacteriophage, bacteria, peptide, protein, antibody, enzyme, amino acid, steroid, drug, carbohydrate, lipid, chromophore, single-stranded or double-stranded nucleic acid, vaccine, and chemical modifications thereof.

37. The method of claim 30, wherein the biological material is a virus or bacteriophage.

38. The method of claim 30, wherein the biological material is an anisotropic particle.

39. The method of claim 30, wherein the biological material is a bacteria.

40. The method of claim 30, wherein the biological material is a peptide or protein.

41. The method of claim 30, wherein at least two biological materials are applied.

42. The method of claim 30, wherein the biological material is an antibody or enzyme.

43. The method of claim 30, wherein the biologic material is layered with an organic compound, inorganic compound, and combinations thereof.

44. The method of claim 30, further comprising the step of applying a storage solution prior to allowing the formation of a stable film.

45. The method of claim 30, further comprising the step of applying a sugar-containing storage solution prior to allowing the formation of a stable film.

46. A kit for fabricating a biofilm storage device comprising:
a container; and
a storage film comprising a biologic material which is stable at room temperature for at least 7 weeks.

47. The kit of claim 46, further comprising a storage solution to be applied to the film.

48. The kit of claim 46, further comprising a sugar-containing storage solution to be applied to the film.

49. The kit of claim 46, further comprising a solvent that promotes film formation.

50. The kit of claim 46, wherein the thin film stores high-density information at room temperature.

51. The kit of claim 50, wherein the high density information is used in diagnosis, screening, analysis, testing, information gathering, data processing, microelectronics, optics, research, or combinations, thereof.

52. The kit of claim 50, wherein the high-density information is stable and chosen from the group consisting of biologic, optical, electrical, magnetic, or combinations, thereof.

53. A hybrid fabricated film storage device comprising:
a substrate comprising a surface; and
a biologic material applied to the surface to form a biologically stable thin film, wherein the film further comprises an inorganic material.

54. The hybrid fabricated film storage device of claim 53, wherein the substrate is further chosen from the group consisting of Langmuir-Bodgett films, functionalized glass, germanium, silicon, a semiconductor material, PTFE, polycarbonate, mica, mylar, plastic, quartz, polystyrene, gallium arsenide, gold, silver, metal, metal alloy, synthetic fabric, and combinations thereof.

55. The hybrid fabricated film storage device of claim 53, wherein the biologically stable thin film is dry.

56. The hybrid fabricated film storage device of claim 53, the substrate further comprises a thin layer which contacts the film of biological material.

57. The hybrid fabricated film storage device of claim 53, wherein film further comprises one or more organic molecules chosen from the group consisting of carbon, single stranded nucleic acid, double stranded nucleic acid, peptide, protein, antibody, enzyme, steroid, drug, chromophore, conducting polymer, or combinations, thereof.

58. The hybrid fabricated film storage device of claim 53, wherein the inorganic material is chosen from the group consisting of indium tin oxide, a doping agent, metal, metal alloy, mineral, or combinations, thereof.

59. The hybrid fabricated film storage device of claim 53, wherein the one or more organic or inorganic molecules are preincubated with the biologic material.

60. The hybrid fabricated film storage device of claim 59, wherein preincubation permits the formation of nanocrystals.

61. The hybrid fabricated film storage device of claim 56, wherein the biologic material is chosen from the group consisting of virus, bacteriophage, bacteria, peptide, protein, amino acid, steroid, drug, chromophore, single-stranded or double-stranded nucleic acid, vaccine, and chemical modifications thereof.

62. The device of claim 56, wherein the biological material is a virus.

63. The device of claim 56, wherein the biological material is a bacteriophage.

64. The device of claim 56, wherein the biological material is bacteria.

65. The device of claim 56, wherein the biological material is peptide or protein.

66. The device of claim 56, wherein the biological material is an antibody.

67. The hybrid fabricated film storage device of claim 56, wherein the biologic material self-assembles to form a uniform thin film.

68. The hybrid fabricated film storage device of claim 56, wherein the biologically stable thin film exhibits biologic, optical, electrical, and magnetic properties, or combinations thereof.

69. The hybrid fabricated film storage device of claim 56, wherein the biologically stable thin film is used in diagnosis, screening, analysis, testing, information gathering, data processing, drug discovery, microelectronics, data storage, research, or combinations thereof.

70. The hybrid fabricated film storage device of claim 56, wherein formation of the biologically stable thin film is controlled by solvent concentration, magnetic field, electric field, optics and combinations thereof.

71. The hybrid fabricated film storage device of claim 56, wherein the biologic material is genetically engineered.

72. The hybrid fabricated film storage device of claim 56, wherein the storage device is stabilized by applying a storage solution to the biologically stable thin film.

73. The hybrid fabricated film storage device of claim 56, wherein the storage device is stabilized by applying a sugar-containing storage solution to the biologically stable thin film.

74. A viral film fabricated for use as a storage device comprising phage particles in a stable film, wherein the film is stable at room temperature for at least 7 weeks.

75. The viral film of claim 74, wherein the stable film is on the surface of a substrate.

76. The viral film of claim 74, wherein the stable film comprises phage particles of a phage display library.

77. The viral film of claim 74, wherein the film comprises micron scale repeating patterns that continue to the centimeter scale.

78. The viral film of claim 74, wherein the film comprises phage particles of a phage display library which preserves ability to infect.

79. The viral film of claim 74, wherein the film has a stable time-to-infection in terms of titer numbers for at least seven weeks.

80. The viral film of claim 74, wherein the film has a stable time-to-infection in terms of titer numbers for at least five months.

81. The viral film of claim 74, wherein the film retains its ability to be greater than 95% infectious for at least 5 months.

82. The viral film of claim 74, wherein the film stores high-density engineered DNA and protein information.

83. The viral film of claim 74, wherein the film is a thin film, having a thickness of about 25 microns of less.

84. The viral film of claim 74, wherein the film is a dry thin film.

85. The viral film of claim 74, wherein the film stores at least 4×10¹³ phage per square centimeter.

86. The viral film of claim 74, further comprising inorganic materials in combination with the phage particles.

87. The viral film of claim 74, further comprising inorganic nanoparticles in combination with the phage particles.

88. The viral film of claim 74, wherein the phage particles are selected to provide for specific binding.

89. The viral film of claim 74, wherein the phage particles are selected to provide for specific binding to inorganic nanoparticles, and phage particles are bound to the inorganic nanoparticles.

90. The viral film of claim 74, wherein the film comprises phage particles of a phage display library, wherein the phage particles are selected to provide for specific binding to inorganic nanoparticles, and phage particles are bound to the inorganic nanoparticles.

91. The viral film of claim 74, wherein the film comprises phage particles of a phage display library, wherein the phage particles are selected to provide for specific binding to biological molecules, and phage particles are bound to the biological molecules.

92. The viral film according to claim 74, wherein the film has a stable time-to-infection in terms of titer numbers for at least seven weeks.

93. Use of the viral film of claim 74 as a storage device in drug discovery, in high throughput screening, or in diagnosis of one or more pathological conditions.

94. A method of forming a viral film comprising:
preparing a concentrated suspension of viral phage particles in a solvent;
removing solvent so that the phage particles form a film under conditions wherein the film is stable at room temperature for at least 7 weeks.

95. The method according to claim 94, wherein the suspension is a liquid crystalline suspension of viral phage particles in the solvent.

96. The method according to claim 94, wherein the substrate is a solid substrate.

97. The method according to claim 94, wherein the film comprises phage particles of a phage display library.

98. The method of claim 94, wherein the film comprises micron scale repeating patterns that continue to the centimeter scale.

99. The method of claim 94, wherein the film comprises phage particles of a phage display library which preserves ability to infect.

100. The method of claim 94, wherein the film has a stable time-to-infection in terms of titer numbers for at least seven weeks.

101. The method of claim 94, wherein the film has a stable time-to-infection in terms of titer numbers for at least five months.

102. The method of claim 94, wherein the film retains its ability to be greater than 95% infectious for at least 5 months.

103. The method of claim 94, wherein the film stores high-density engineered DNA and protein information.

104. The method of claim 94, wherein the film is a thin film.

105. The method of claim 94, wherein the film is a dry thin film.

106. The method of claim 94, wherein the film stores at least 4×10¹³ phage per square centimeter.

107. The method of claim 94, wherein the film further comprises inorganic compounds in combination with the phage particles.

108. The method of claim 94, wherein the film further comprises inorganic nanoparticles in combination with the phage particles, and the film retains its ability to be greater than 95% infectious for at least 5 months.

109. The method of claim 94, wherein the phage particles are selected phage particles to provide for specific binding.

110. The method of claim 94, wherein the phage particles are selected phage particles to provide for specific binding to inorganic nanoparticles, and the phage particles are bound to the inorganic nanoparticles.

111. The method of claim 94, wherein the phage particles are selected phage particles to provide for specific binding to biological molecules, and the phage particles are bound to the biological molecules.

112. A self-supporting film for use as a storage device comprising one or more biological materials, wherein the film is stable for at least six months.

113. The film according to claim ill, wherein the one or more biological materials is self-assembled to form a thin film on the contacting surface of a substrate.

114. The film according to claim 111, wherein the film is liquid crystalline.

115. The film according to claim 111, wherein the biological material is a virus.

116. The film according to claim 111, wherein the biological material is a bacteriophage.

117. The film according to claim 111, wherein the biological material is an enzyme.

118. The film according to claim 111, wherein the biological material is a peptide or protein.

119. The film according to claim 111, wherein the film further comprises an inorganic nanoparticle.

120. The film according to claim 111, wherein the film further comprises an inorganic nanoparticle which is specifically bound to the biological material.

121. The film according to claim 111, wherein the biological material is a peptide.

122. A method for improving the stability and long term activity of a biofilm storage device comprising the step of including a storage solution in the biofilm storage device which improves the stability and long term activity of the biofilm storage device.

123. The method according to claim 122, wherein the storage solution comprises sugar.

124. The method according to claim 122, wherein the storage device comprises an enzyme.

125. The method according to claim 122, wherein the storage devices comprises an enzyme and a virus.

126. A method to visualize the structure and function of a biological material used as a biofilm storage device, comprising the step of monitoring light properties of the biological material.

127. The method of claim 126, wherein the light-emitting molecule is a protein.

128. The method of claim 126, wherein the light properties are monitored by confocal microscopy.

129. The method of claim 126, wherein the light-emitting molecules are fluorescent.

130. A method of forming viral thin films for a storage device which retain the ability of the viral particles to infect a bacterial host, comprising the step of removing solvent from a concentrated suspension of viral particles to form the viral thin film on a substrate, wherein the viral particles retain infecting ability for a bacterial host based on measurement of titer numbers after at least seven weeks.

131. The method according to claim 130, wherein the infecting ability is based on measurement of titer numbers after at least five months.

132. The method according to claim 130, wherein the viral particles form epitaxial layer domains on the substrate.

133. The method according to claim 130, wherein the thin film stores at least 4×10¹³ phage per square centimeter.

134. The method according to claim 130, wherein the thin film stores at least 7200 times 4×10¹³ protein units per square centimeter.

135. The method according to claim 130, wherein the viral particles comprise a filamentous phage virus.

136. The method according to claim 130, wherein the viral particles before film formation comprise a genetically engineered phage library, and the library information is preserved in film form.

137. The method according to claim 130, wherein the viral particles are designed to provide the film with specific binding properties so that the film can be a storage device for input and output of information.

138. A storage device comprising liquid crystalline viral film comprising anisotropic viral particles which are in the chiral smectic C phase.

139. The storage device according to claim 138, wherein the viral particles are a phage display library.

140. A storage device according to claim 138, wherein the viral particles are genetically engineered.

141. A storage device according to claim 138, wherein the viral particles are selected to specifically bind to an organic or inorganic compound.

142. A storage device according to claim 138, wherein the film has a thickness of about one micron to about 25 microns.

143. A storage device according to claim 138, wherein the film further comprises inorganic nanoparticles.

144. A storage device according to claim 138, wherein the film further comprises a stabilization agent.

145. A storage device according to claim 138, wherein the film further comprises a biomaterial.

146. A storage device according to claim 138, wherein the film further comprises a biomaterial and inorganic nanoparticles.

147. A method of making a storage device comprising the step of casting a film of viral particles under concentration conditions which provide for a chiral smectic C phase in the film.

148. The method acccording to claim 147, wherein the concentration of viral particles is at least about 1 mg/mL.

149. The method according to claim 147, wherein the concentration of viral partilcles is sufficiently high to provide a self-supporting film.

150. A storage device comprising a viral film which has been selected to bind streptavidin protein units.

151. The storage device according to claim 150, wherein the viral film further comprises metallic nanoparticles.

152. The storage device according to claim 150, wherein the viral film further comprises fluorescent molecules.

153. The storage device according to claim 150, wherein the viral film further comprises fluorescent protein.

154. The storage device according to claim 150, wherein the film is liquid crystalline.

155. The storage device according to claim 150, wherein the film further comprises a stabilization agent.

156. A method of forming a storage device comprising providing a phage display library and by panning to select phage which specifically bind to streptavidin.

157. The method according to claim 156, further comprising the step of binding the selected phage to an inorganic nanoparticle having streptavidin units.

158. The method according to claim 156, further comprising the step of binding the selected phage to a fluorescent compound having streptavidin units.

159. The method according to claim 156, further comprising the step of binding the selected phage to a fluorescent protein having streptavidin units.


SEQUENCE LISTING (116 sequences)

SEQ ID NO: 1 - protein, 12 aa, Artificial Sequence
  Description of Artificial Sequence Synthetic peptide
AMAGTTSDPSTV

SEQ ID NO: 2 - protein, 12 aa, Artificial Sequence
  Description of Artificial Sequence Synthetic peptide
PAQSMSQTPSAA

SEQ ID NO: 3 - protein, 12 aa, Artificial Sequence
  Description of Artificial Sequence Synthetic peptide
HTHTNNDSPNQA

SEQ ID NO: 4 - protein, 12 aa, Artificial Sequence
  Description of Artificial Sequence Synthetic peptide
DTQGFHSRSSSA

SEQ ID NO: 5 - protein, 12 aa, Artificial Sequence
  Description of Artificial Sequence Synthetic peptide
TSSSALQPAHAW

SEQ ID NO: 6 - protein, 12 aa, Artificial Sequence
  Description of Artificial Sequence Synthetic peptide
SESSPISLDYRA

SEQ ID NO: 7 - protein, 12 aa, Artificial Sequence
  Description of Artificial Sequence Synthetic peptide
STHNYQIPRPPT

SEQ ID NO: 8 - protein, 12 aa, Artificial Sequence
  Description of Artificial Sequence Synthetic peptide
HPFSNEPLQLSS

SEQ ID NO: 9 - protein, 12 aa, Artificial Sequence
  Description of Artificial Sequence Synthetic peptide
SSLFIQQNALTG

SEQ ID NO: 10 - protein, 12 aa, Artificial Sequence
  Description of Artificial Sequence Synthetic peptide
GPFPTMPLPNGH

SEQ ID NO: 11 - protein, 12 aa, Artificial Sequence
  Description of Artificial Sequence Synthetic peptide
GSGQLPIALELR

SEQ ID NO: 12 - protein, 9 aa, Artificial Sequence
  Description of Artificial Sequence Synthetic peptide
CHASNRLSC

SEQ ID NO: 13 - protein, 12 aa, Artificial Sequence
  Description of Artificial Sequence Synthetic peptide
SMDRSDMTMRLP

SEQ ID NO: 14 - protein, 12 aa, Artificial Sequence
  Description of Artificial Sequence Synthetic peptide
GTFTPRPTPIYP

SEQ ID NO: 15 - protein, 12 aa, Artificial Sequence
  Description of Artificial Sequence Synthetic peptide
QMSENLTSQIES

SEQ ID NO: 16 - protein, 12 aa, Artificial Sequence
  Description of Artificial Sequence Synthetic peptide
DMLARLRATAGP

SEQ ID NO: 17 - protein, 12 aa, Artificial Sequence
  Description of Artificial Sequence Synthetic peptide
SQTWLLMSPVAT

SEQ ID NO: 18 - protein, 12 aa, Artificial Sequence
  Description of Artificial Sequence Synthetic peptide
ASPDQQVGPLYV

SEQ ID NO: 19 - protein, 12 aa, Artificial Sequence
  Description of Artificial Sequence Synthetic peptide
LTWSPLQTVARF

SEQ ID NO: 20 - protein, 12 aa, Artificial Sequence
  Description of Artificial Sequence Synthetic peptide
QISAHQMPSRPI

SEQ ID NO: 21 - protein, 12 aa, Artificial Sequence
  Description of Artificial Sequence Synthetic peptide
SMKYNLIVDSPY

SEQ ID NO: 22 - protein, 12 aa, Artificial Sequence
  Description of Artificial Sequence Synthetic peptide
QMPIRNQLAWPM

SEQ ID NO: 23 - protein, 12 aa, Artificial Sequence
  Description of Artificial Sequence Synthetic peptide
TQNLEIREPLTP

SEQ ID NO: 24 - protein, 12 aa, Artificial Sequence
  Description of Artificial Sequence Synthetic peptide
YPMSPSPYPYQL

SEQ ID NO: 25 - protein, 12 aa, Artificial Sequence
  Description of Artificial Sequence Synthetic peptide
SFMIQPTPLPPS

SEQ ID NO: 26 - protein, 12 aa, Artificial Sequence
  Description of Artificial Sequence Synthetic peptide
GLAPHIHSLNEA

SEQ ID NO: 27 - protein, 12 aa, Artificial Sequence
  Description of Artificial Sequence Synthetic peptide
MQFPVTPYLNAS

SEQ ID NO: 28 - protein, 12 aa, Artificial Sequence
  Description of Artificial Sequence Synthetic peptide
SPGDSLKKLAAS

SEQ ID NO: 29 - protein, 12 aa, Artificial Sequence
  Description of Artificial Sequence Synthetic peptide
GYHMQTLPGPVA

SEQ ID NO: 30 - protein, 12 aa, Artificial Sequence
  Description of Artificial Sequence Synthetic peptide
SLTPLTTSHLRS

SEQ ID NO: 31 - protein, 12 aa, Artificial Sequence
  Description of Artificial Sequence Synthetic peptide
TLTNGPLRPFTG

SEQ ID NO: 32 - protein, 12 aa, Artificial Sequence
  Description of Artificial Sequence Synthetic peptide
LNTPKPFTLGQN

SEQ ID NO: 33 - protein, 9 aa, Artificial Sequence
  Description of Artificial Sequence Synthetic peptide
CDLQNYKAC

SEQ ID NO: 34 - protein, 9 aa, Artificial Sequence
  Description of Artificial Sequence Synthetic peptide
CRHPHTRLC

SEQ ID NO: 35 - protein, 9 aa, Artificial Sequence
  Description of Artificial Sequence Synthetic peptide
CANLKPKAC

SEQ ID NO: 36 - protein, 9 aa, Artificial Sequence
  Description of Artificial Sequence Synthetic peptide
CYINPPKVC

SEQ ID NO: 37 - protein, 9 aa, Artificial Sequence
  Description of Artificial Sequence Synthetic peptide
CNNKVPVLC

SEQ ID NO: 38 - protein, 9 aa, Artificial Sequence
  Description of Artificial Sequence Synthetic peptide
CHASKTPLC

SEQ ID NO: 39 - protein, 9 aa, Artificial Sequence
  Description of Artificial Sequence Synthetic peptide
CASQLYPAC

SEQ ID NO: 40 - protein, 9 aa, Artificial Sequence
  Description of Artificial Sequence Synthetic peptide
CNMTQYPAC

SEQ ID NO: 41 - protein, 9 aa, Artificial Sequence
  Description of Artificial Sequence Synthetic peptide
CFAPSGPAC

SEQ ID NO: 42 - protein, 9 aa, Artificial Sequence
  Description of Artificial Sequence Synthetic peptide
CPVWIQAPC

SEQ ID NO: 43 - protein, 9 aa, Artificial Sequence
  Description of Artificial Sequence Synthetic peptide
CQVAVNPLC

SEQ ID NO: 44 - protein, 9 aa, Artificial Sequence
  Description of Artificial Sequence Synthetic peptide
CQPEAMPAC

SEQ ID NO: 45 - protein, 9 aa, Artificial Sequence
  Description of Artificial Sequence Synthetic peptide
CHPTMPLAC

SEQ ID NO: 46 - protein, 9 aa, Artificial Sequence
  Description of Artificial Sequence Synthetic peptide
CPPFAAPIC

SEQ ID NO: 47 - protein, 9 aa, Artificial Sequence
  Description of Artificial Sequence Synthetic peptide
CNKHQPMHC

SEQ ID NO: 48 - protein, 9 aa, Artificial Sequence
  Description of Artificial Sequence Synthetic peptide
CFPMRSNQC

SEQ ID NO: 49 - protein, 9 aa, Artificial Sequence
  Description of Artificial Sequence Synthetic peptide
CQSMPHNRC

SEQ ID NO: 50 - protein, 9 aa, Artificial Sequence
  Description of Artificial Sequence Synthetic peptide
CNNPMHQNC

SEQ ID NO: 51 - protein, 9 aa, Artificial Sequence
  Description of Artificial Sequence Synthetic peptide
CHMAPRWQC

SEQ ID NO: 52 - protein, 9 aa, Artificial Sequence
  Description of Artificial Sequence Synthetic peptide
HVHIHSRPM

SEQ ID NO: 53 - protein, 9 aa, Artificial Sequence
  Description of Artificial Sequence Synthetic peptide
LPNMHPLPL

SEQ ID NO: 54 - protein, 9 aa, Artificial Sequence
  Description of Artificial Sequence Synthetic peptide
LPLRLPPMP

SEQ ID NO: 55 - protein, 9 aa, Artificial Sequence
  Description of Artificial Sequence Synthetic peptide
HSMIGTPTT

SEQ ID NO: 56 - protein, 9 aa, Artificial Sequence
  Description of Artificial Sequence Synthetic peptide
SVSVGMKPS

SEQ ID NO: 57 - protein, 9 aa, Artificial Sequence
  Description of Artificial Sequence Synthetic peptide
LDASFMQDW

SEQ ID NO: 58 - protein, 9 aa, Artificial Sequence
  Description of Artificial Sequence Synthetic peptide
TPPSYQMAM

SEQ ID NO: 59 - protein, 9 aa, Artificial Sequence
  Description of Artificial Sequence Synthetic peptide
YPQLVSMST

SEQ ID NO: 60 - protein, 9 aa, Artificial Sequence
  Description of Artificial Sequence Synthetic peptide
GYSTINMYS

SEQ ID NO: 61 - protein, 9 aa, Artificial Sequence
  Description of Artificial Sequence Synthetic peptide
DRMLLPFNL

SEQ ID NO: 62 - protein, 9 aa, Artificial Sequence
  Description of Artificial Sequence Synthetic peptide
IPMTPSYDS

SEQ ID NO: 63 - protein, 9 aa, Artificial Sequence
  Description of Artificial Sequence Synthetic peptide
MYSPRPPAL

SEQ ID NO: 64 - protein, 9 aa, Artificial Sequence
  Description of Artificial Sequence Synthetic peptide
QPTTDLMAH

SEQ ID NO: 65 - protein, 9 aa, Artificial Sequence
  Description of Artificial Sequence Synthetic peptide
ATHVQMAWA

SEQ ID NO: 66 - protein, 9 aa, Artificial Sequence
  Description of Artificial Sequence Synthetic peptide
SMHATLTPM

SEQ ID NO: 67 - protein, 9 aa, Artificial Sequence
  Description of Artificial Sequence Synthetic peptide
SGPAHGMFA

SEQ ID NO: 68 - protein, 9 aa, Artificial Sequence
  Description of Artificial Sequence Synthetic peptide
IANRPYSAQ

SEQ ID NO: 69 - protein, 7 aa, Artificial Sequence
  Description of Artificial Sequence Synthetic peptide
VMTQPTR

SEQ ID NO: 70 - protein, 7 aa, Artificial Sequence
  Description of Artificial Sequence Synthetic peptide
HMRPLSI

SEQ ID NO: 71 - protein, 12 aa, Artificial Sequence
  Description of Artificial Sequence Synthetic peptide
LTRSPLHVDQRR

SEQ ID NO: 72 - protein, 12 aa, Artificial Sequence
  Description of Artificial Sequence Synthetic peptide
VISNHAESSRRL

SEQ ID NO: 73 - protein, 7 aa, Artificial Sequence
  Description of Artificial Sequence Synthetic peptide
HTHIPNQ

SEQ ID NO: 74 - protein, 7 aa, Artificial Sequence
  Description of Artificial Sequence Synthetic peptide
LAPVSPP

SEQ ID NO: 75 - protein, 9 aa, Artificial Sequence
  Description of Artificial Sequence Synthetic peptide
CMTAGKNTC

SEQ ID NO: 76 - protein, 9 aa, Artificial Sequence
  Description of Artificial Sequence Synthetic peptide
CQTLWRNSC

SEQ ID NO: 77 - protein, 9 aa, Artificial Sequence
  Description of Artificial Sequence Synthetic peptide
CTSVHTNTC

SEQ ID NO: 78 - protein, 9 aa, Artificial Sequence
  Description of Artificial Sequence Synthetic peptide
CPSLAMNSC

SEQ ID NO: 79 - protein, 9 aa, Artificial Sequence
  Description of Artificial Sequence Synthetic peptide
CSNNTVHAC

SEQ ID NO: 80 - protein, 9 aa, Artificial Sequence
  Description of Artificial Sequence Synthetic peptide
CLPAQGHVC

SEQ ID NO: 81 - protein, 9 aa, Artificial Sequence
  Description of Artificial Sequence Synthetic peptide
CLPAQVHVC

SEQ ID NO: 82 - protein, 9 aa, Artificial Sequence
  Description of Artificial Sequence Synthetic peptide
CPPKNVRLC

SEQ ID NO: 83 - protein, 9 aa, Artificial Sequence
  Description of Artificial Sequence Synthetic peptide
CPHINAHAC

SEQ ID NO: 84 - protein, 9 aa, Artificial Sequence
  Description of Artificial Sequence Synthetic peptide
CIVNLARAC

SEQ ID NO: 85 - protein, 12 aa, Artificial Sequence
  Description of Artificial Sequence Synthetic peptide
TMGFTAPRFPHY

SEQ ID NO: 86 - protein, 12 aa, Artificial Sequence
  Description of Artificial Sequence Synthetic peptide
ATQSYVRHPSLG

SEQ ID NO: 87 - protein, 12 aa, Artificial Sequence
  Description of Artificial Sequence Synthetic peptide
TSTTQGALAYLF

SEQ ID NO: 88 - protein, 12 aa, Artificial Sequence
  Description of Artificial Sequence Synthetic peptide
DPPWSAIVRHRD

SEQ ID NO: 89 - protein, 12 aa, Artificial Sequence
  Description of Artificial Sequence Synthetic peptide
FDNKPFLRVASE

SEQ ID NO: 90 - protein, 12 aa, Artificial Sequence
  Description of Artificial Sequence Synthetic peptide
HQSHTQQNKRHL

SEQ ID NO: 91 - protein, 12 aa, Artificial Sequence
  Description of Artificial Sequence Synthetic peptide
TSTTQGALAYLF

SEQ ID NO: 92 - protein, 12 aa, Artificial Sequence
  Description of Artificial Sequence Synthetic peptide
KTPIHTSAWEFQ

SEQ ID NO: 93 - protein, 12 aa, Artificial Sequence
  Description of Artificial Sequence Synthetic peptide
DLFHLKPVSNEK

SEQ ID NO: 94 - protein, 12 aa, Artificial Sequence
  Description of Artificial Sequence Synthetic peptide
KPFWTSSPDVMT

SEQ ID NO: 95 - protein, 12 aa, Artificial Sequence
  Description of Artificial Sequence Synthetic peptide
PWAATSKPPYSS

SEQ ID NO: 96 - protein, 12 aa, Artificial Sequence
  Description of Artificial Sequence Synthetic peptide
WQSELXXASNLP

SEQ ID NO: 97 - protein, 12 aa, Artificial Sequence
  Description of Artificial Sequence Synthetic peptide
AEATEARPYLRA

SEQ ID NO: 98 - protein, 12 aa, Artificial Sequence
  Description of Artificial Sequence Synthetic peptide
AYHNSGKTKTET

SEQ ID NO: 99 - protein, 12 aa, Artificial Sequence
  Description of Artificial Sequence Synthetic peptide
SPITPPLPPLPE

SEQ ID NO: 100 - protein, 12 aa, Artificial Sequence
  Description of Artificial Sequence Synthetic peptide
ETNLGPQPYPVR

SEQ ID NO: 101 - protein, 12 aa, Artificial Sequence
  Description of Artificial Sequence Synthetic peptide
SQLYNTPPQTAV

SEQ ID NO: 102 - protein, 12 aa, Artificial Sequence
  Description of Artificial Sequence Synthetic peptide
TGHHIHLQAHPI

SEQ ID NO: 103 - protein, 12 aa, Artificial Sequence
  Description of Artificial Sequence Synthetic peptide
VPQIPNLISHPM

SEQ ID NO: 104 - protein, 12 aa, Artificial Sequence
  Description of Artificial Sequence Synthetic peptide
WELPWIDSNHPQ

SEQ ID NO: 105 - protein, 11 aa, Artificial Sequence
  Description of Artificial Sequence Synthetic peptide
IQSTFTLHPWV

SEQ ID NO: 106 - protein, 11 aa, Artificial Sequence
  Description of Artificial Sequence Synthetic peptide
KPYLFLQPNYG

SEQ ID NO: 107 - protein, 11 aa, Artificial Sequence
  Description of Artificial Sequence Synthetic peptide
NGHVHLPAHPQ

SEQ ID NO: 108 - protein, 12 aa, Artificial Sequence
  Description of Artificial Sequence Synthetic peptide
EYTHPLLLAHPI

SEQ ID NO: 109 - protein, 12 aa, Artificial Sequence
  Description of Artificial Sequence Synthetic peptide
LPVNAWLVSHPQ

SEQ ID NO: 110 - protein, 12 aa, Artificial Sequence
  Description of Artificial Sequence Synthetic peptide
QPSWSLLLEHPH

SEQ ID NO: 111 - protein, 12 aa, Artificial Sequence
  Description of Artificial Sequence Synthetic peptide
AAKATLSGTASV

SEQ ID NO: 112 - protein, 12 aa, Artificial Sequence
  Description of Artificial Sequence Synthetic peptide
QDPYSHLLQHPQ

SEQ ID NO: 113 - protein, 12 aa, Artificial Sequence
  Description of Artificial Sequence Synthetic peptide
TTXFPWLQTHPQ

SEQ ID NO: 114 - protein, 12 aa, Artificial Sequence
  Description of Artificial Sequence Synthetic peptide
QNWTWSLPHHPQ

SEQ ID NO: 115 - protein, 12 aa, Artificial Sequence
  Description of Artificial Sequence Synthetic peptide
WDPYSHLLQHPQ

SEQ ID NO: 116 - protein, 12 aa, Artificial Sequence
  Description of Artificial Sequence Synthetic peptide
IGSRAETMPWPR